(19) United States
(12) Patent Application Publication
(10) Pub. No.: US 2022/0076800 A1
(43) Pub. Date: Mar. 10, 2022

(54) Subject Modelling

(71) Applicant: Neurotech Research Pty. Ltd., Brisbane (AU)

(72) Inventor: Nigel Greenwood, Brisbane (AU)

(21) Appl. No.: 17/408,247

(22) Filed: Aug. 20, 2021

Related U.S. Application Data
(63) Continuation-in-part of application No. 12/530,533, filed on Nov. 9, 2009, now abandoned.
(60) Provisional application No. 60/782,560, filed on Mar. 15, 2006.

(30) Foreign Application Priority Data
Mar. 10, 2006 (AU) 2006901231

(51) Int. Cl. G16H 20/17; G06F 30/20; A61K 38/28; A61K 31/7004; G16B 5/00

(52) U.S. Cl. CPC G16H 20/17; G06F 30/20; G06F 2111/10; A61K 31/7004; G16B 5/00; A61K 38/28

(57) ABSTRACT

A method of modelling the biological response of a biological subject. The method includes, in a processing system, for a model including one or more equations and associated parameters, comparing at least one measured subject attribute and at least one corresponding model value. The model is then modified in accordance with results of the comparison to thereby more effectively model the biological response.

### BACKGROUND OF THE INVENTION

The present invention relates to a method and apparatus for use in modelling the biological response of a biological subject, and in particular to a method and apparatus that can be used for generating a model representing the effect of one or more conditions on a living body.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Currently, it is known to determine medication programs to allow drugs to be administered for different medical conditions. However the determination of the drug programs typically requires years of experimentation. Even then the regimes are typically fairly simplistic and rely on the patient taking specified quantities of medication at various time intervals.

WO2004027674 describes a process for using a derived model to allow treatment program for a subject. However, current techniques for model development are limited.

Model Reference Adaptive Control (MRAC) is a technique used for controlling physical objects in accordance with predictive models. In particular, the process involves taking a complicated bit of machinery or electronics, often referred to as a “plant”, that is not easily modelled, and constraining its behaviour so that it behaves in a manner that is increasingly similar to a theoretical model that the control process uses as its “reference”.

This is commonly used for example to allow robot arms to be controlled using appropriate control algorithms. In this instance, each robot arm will react slightly differently to set commands, due to variations in physical and environmental factors, such as gear wear, or the like. To overcome this, MRAC operates by using a reference model of a robot arm and monitoring the operation of the actual arm for specified control commands, and then uses sensor feedback of this ensuing change of state to modify the control commands and/or model parameters, altering the arm's response, to ensure that there is concordance between the stipulated model behaviour and actual arm operation, when commanded by the control algorithms.

Within the field of MRAC as applied to robotics or electrical engineering, variations on the basic idea include “Identification” in an adaptive control context. If the reference model is sufficiently close in mathematical structure to that of the unknown robot dynamics, the process of modifying the model parameters to ensure concordance between the stipulated model behaviour and actual arm operation can be regarded as a process of estimating actual arm parameters within the context of the model structure. In its most extreme form, identification-based algorithms can be regarded as an “inversion” of conventional MRAC techniques, so that the model comes to track the plant rather than vice versa.

Woo, J. and Rootenberg J., (1975) Lyapunov Redesign of Model Reference Adaptive Control System for Long Term Ventilation of Lung, ISA Trans.; 14(1):89-98 describes conventional application of MRAC to an “iron lung” to regulate ventilation, and therefore is limited to application with a specific hardware configuration.

Palerm, C. C. R., Drug Infusion Control: an Extended Direct Model Reference Adaptive Control Strategy describes basic linear MRAC, mainly as applied to drug administration for diabetes. However, this is limited to a linear MRAC scheme and as biological systems are generally non-linear, this has limited application in generalised biological scenarios.

WO2004027674 provides a method of determining a treatment program for a subject. The method includes obtaining subject data representing the subject's condition. The subject data is used together with a model of the condition, to determine system values representing the condition. These system values are then used to determining one or more trajectories representing the progression of the condition in accordance with the model. From this, it is possible to determine a treatment program in accordance with the determined trajectories.

### SUMMARY OF THE PRESENT INVENTION

In a first broad form the present invention provides a method of modelling the biological response of a biological subject, the method including, in a processing system: for a model including one or more equations and associated parameters, comparing at least one measured subject attribute and at least one corresponding model value; and, modifying the model in accordance with results of the comparison to thereby more effectively model the biological response.

Typically the method includes: determining a difference between the at least one measured subject attribute and the at least one corresponding model value; and, modifying the model in accordance with the determined difference.

Typically the method includes, in the processing system, iteratively modifying the model until at least one of: the difference is below a predetermined threshold; the difference asymptotically approaches an acceptable limit; and, the difference is minimised.

Typically the method includes, in the processing system: determining a subject trajectory representing changes in the at least one measured subject attribute over time; determining a model trajectory representing changes in the at least one corresponding model value over time; and, performing the comparison by comparing the trajectories.

Typically the method includes, in the processing system, iteratively modifying the model until the model and subject trajectories converge.

Typically the method includes: using control inputs to induce at least one of a perturbation and agitation of the subject into a non-equilibrium condition; and, determining at least one measured subject attribute under the non-equilibrium condition.

Typically the method includes, in the processing system: forming a linear error equation representing a difference between a desired state of the subject and an actual state; and, constructing a control algorithm to minimise the error equation.

Typically the method includes, in the processing system, at least one of: using Lyapunov stability methods to ensure convergence of subject and model behaviour through use of one or more Lyapunov functions; and, using a derivative of one or more Lyapunov functions to impose convergence of subject and model behaviour.

Typically the method includes, in the processing system, modifying the model using at least one of: model reference adaptive control-based methods; Lyapunov stability-based methods; and, in the event that the subject exhibits mathematically chaotic behaviour, using data obtained from surface-of-section embedding techniques.

Typically the method includes, in the processing system: determining a Lyapunov function; determining a numerical value of a derivative of a Lyapunov function, and using the Lyapunov function to modify at least one model value.

Typically the method includes, in the processing system, at least one of the following: using the existence of a Lyapunov function as the mathematical basis for employing other algorithms to modify at least one model value; and, in the case of chaotic behaviour being exhibited by the subject, using surface-of-section embedding techniques as the mathematical basis for employing other algorithms to modify at least one model value.

Typically the method includes, in the processing system, at least one of: using pattern-finding or optimisation algorithms to at least one of: select one of a number of predetermined Lyapunov functions; and/or, optimise a Lyapunov function; and/or optimise the derivative of a Lyapunov function, searching candidate Lyapunov functions to determine a function resulting in the best improvement to the model; and, at least one of: searching the derivatives of candidate Lyapunov functions to determine a function resulting in the best improvement to the model; and employing candidate derivatives without explicitly invoking the underlying Lyapunov function.

Typically the method includes, in the processing system, using pattern-finding or optimisation algorithms to determine a function or related algorithms resulting in the best improvement to the model.

Typically the model is formed from at least one non-linear ordinary differential equation or difference equation.

Typically the model value includes at least one of: State variable values representing rapidly changing attributes; Parameter values representing slowly changing or constant attributes; and, Control variable values representing attributes of the biological response that can be externally controlled.

Typically the method includes, in the processing system in the instance of mathematically-chaotic behaviour being exhibited by the subject, at least one of the following: using the data obtained from surface-of-section embedding techniques to determine an improvement in the model within the domain of chaotic behaviour, by modifying at least one of the following: At least one equation; and, At least one model value; and, using the data obtained from surface-of-section embedding techniques, to determine an improvement in the model outside the domain of chaotic behaviour, by modifying at least one of the following: At least one equation; and, At least one model value.

Typically the method includes, in the processing system: determining a condition-independent base model; and, updating the base model to determine a condition-specific model by modifying at least one of: at least one equation; and, at least one model value.

Typically the method includes, in the processing system: selecting a base model from a number of predetermined base models; and, modifying the model to thereby simulate a condition within the subject.

Typically the base model is formed from at least one of: biological components; pharmacological components; pharmacodynamic components; and, pharmacokinetic components.

Typically the measured subject attribute is the subject status and the model value is a model output value indicative of the modelled subject status.

Typically the subject is at least one of a patient, an animal or an in vitro tissue culture.

Typically the model models a condition including at least one of: Degenerative diseases such as Parkinson's or Alzheimer's; Disorders involving dopaminergic neurons; Schizophrenia; Bipolar disorders/manic depression; Cardiac disorders; Myasthenia gravis; Neuro-muscular disorders; Cancerous and tumorous cells and related disorders; HIV/AIDS and other immune or auto-immune system disorders; Hepatic disorders; Athletic conditioning; Pathogen related conditions; Viral, bacterial or other infectious diseases; Leukemia; Poisoning, including snakebite and other venom-based disorders; Insulin-dependent diabetes; Clinical trialing of drugs; Any other instances of medication or drug administration to a subject, such that repeated doses are administered over time to maintain drug or ligand concentration to a desired level or within an interval of levels, in the presence of dissipative pharmacokinetic processes such as those of uptake or absorption, distribution or transport, metabolism or elimination; Reconstruction of cardiac rhythms, function, arrhythmia or other cardiac output; Drug-based control of arterial pressure.

Typically the method includes, in the processing system, using the model to perform at least one of: determining a health status of the subject; diagnosing a presence, absence or degree of a condition; treating a condition; and, determining at least one biological attribute for the subject.

In a second broad form the present invention provides apparatus for modelling the biological response of a biological subject, the apparatus including a processing system for: for a model including one or more equations and associated parameters, comparing at least one measured subject attribute and at least one corresponding model value; and, modifying the model in accordance with results of the comparison to thereby more effectively model the biological response.

In a third broad form the present invention provides a computer program product for modelling the biological response of a biological subject, the computer program product being formed from computer executable code, which when executed using a suitable processing system causes the processing system to: for a model including one or more equations and associated parameters, compare at least one measured subject attribute and at least one corresponding model value; and, modify the model in accordance with results of the comparison to thereby more effectively model the biological response.

In a fourth broad form the present invention provides a method for use in at least one of treating or diagnosing a subject, the method including modelling a biological response of a biological subject, using a processing system that: for a model including one or more equations and associated parameters, compares at least one measured subject attribute and at least one corresponding model value; modifies the model in accordance with results of the comparison to thereby more effectively model the biological response; and, using the model to at least one of treat and diagnose a condition within the subject.

In one broad form, an aspect of the present invention seeks to provide a method of treating diabetes within a biological subject, the method comprising, in a processing system having a processor and a memory: a) using the processing system to determine measured subject attributes of the biological subject, the measured subject attributes being periodically measured over a time period and wherein the measured subject attributes include a glucose attribute at least partially indicative of blood glucose levels; b) using the processing system to determine a base model including one or more equations including at least one non-linear ordinary differential equation or difference equation and wherein the model includes associated variables and parameters including: one or more state variables representing rapidly changing subject attributes, and including the glucose attribute; one or more parameters representing slowly changing or constant subject attributes, and including at least one of: a glucose disappearance rate constant; an insulin disappearance rate constant; a plasma glucose concentration increase for a given dosage of glucose administered to the subject; and, a plasma insulin concentration increase for a given dosage of insulin administered to the subject; and, one or more control variables representing attributes of a biological response of the subject that can be externally controlled using control inputs provided to the subject, and wherein at least one control input represents treatment that can be provided to the subject, the one or more control variables including at least one dosage of insulin administered to the subject; c) using the processor of the processing system to calculate at least one model value using the base model, the at least one model value being a value of at least one of a state variable, a control variable and a parameter; d) using the processor of the processing system to compare the measured subject attributes and at least one corresponding model value to determine a difference between the measured subject attributes and the at least one corresponding model value, the difference representing an accuracy of the base model, and the difference being determined by having the processor: i) derive a subject trajectory representing changes in the measured subject attributes over the time period; ii) calculate a model trajectory representing changes in the at least one corresponding model value over the time period; and, iii) perform the comparison by comparing the subject and model trajectories; e) using the processor of the processing system to modify the base model in accordance with the determined difference, to improve said accuracy of the base model, the base model being modified by modifying at least one of: i) at least one equation; and, ii) at least one model value; f) using the processor of the processing system to repeat steps c) to e) to iteratively modify the base model to thereby generate a subject model representing the condition, the iterative modifying being performed until at least one of: i) the difference is below a predetermined threshold; ii) the difference asymptotically approaches an acceptable limit; and, iii) the difference is minimised; g) using the subject model to treat a condition within the subject by: using the model to derive a treatment regime including at least one dosage of a substance to be administered to the subject; and administering the at least one dose of the substance to the subject in accordance with the treatment regime to thereby treat the subject, and wherein the substance is at least one of insulin and glucose.

Typically the method includes, in the processor of the processing system, iteratively modifying the base model until the model and subject trajectories converge.

Typically the iterative process includes: using external control inputs by administering treatment to the subject to induce a perturbation or agitation of a status of the subject; and, determining at least one measured subject attribute whilst the perturbation or agitation is induced so that the model can be modified to simulate the application of the control inputs; and,

Typically the method includes: a) using control inputs by administering treatment to the subject to induce at least one of a perturbation and agitation of the subject into a non-equilibrium condition; and, b) determining at least one measured subject attribute under the non-equilibrium condition.

Typically the method includes, in the processor of the processing system, using the model to derive a treatment regime by: a) forming a linear error equation representing a difference between a desired state of the subject and an actual state; and, b) constructing a control algorithm including control variable values that minimise the error equation.

Typically the method includes, in the processor of the processing system, at least one of: a) using Lyapunov stability methods to ensure convergence of subject and model behaviour through use of one or more Lyapunov functions; and, b) using a derivative of one or more Lyapunov functions to impose convergence of subject and model behaviour.

Typically the method includes, in the processor of the processing system, modifying the base model using at least one of: a) model reference adaptive control-based methods; b) Lyapunov stability-based methods; and, c) in the event that the subject exhibits mathematically chaotic behaviour, using data obtained from surface-of-section embedding techniques.

Typically the method includes, in the processor of the processing system: a) determining a Lyapunov function; b) determining a numerical value of a derivative of a Lyapunov function, and c) using the Lyapunov function to modify at least one model value.

Typically the method includes, in the processor of the processing system, at least one of the following: a) using the existence of a Lyapunov function as the mathematical basis for employing other algorithms to modify at least one model value; and, b) in the case of chaotic behaviour being exhibited by the subject, using surface-of-section embedding techniques as the mathematical basis for employing other algorithms to modify at least one model value.

Typically the method includes, in the processor of the processing system, at least one of: a) using pattern-finding or optimisation algorithms to at least one of: i) select one of a number of predetermined Lyapunov functions; and/or, ii) optimise a Lyapunov function; and/or iii) optimise the derivative of a Lyapunov function, b) searching candidate Lyapunov functions to determine a function resulting in the best improvement to the base model; and, c) at least one of: i) searching the derivatives of candidate Lyapunov functions to determine a function resulting in the best improvement to the base model; and ii) employing candidate derivatives without explicitly invoking the underlying Lyapunov function.

Typically the method includes, in the processor of the processing system, using pattern-finding or optimisation algorithms to determine a function or related algorithms resulting in the best improvement to the base model.

Typically the method includes, in the processor of the processing system and in the instance of mathematically-chaotic behaviour being exhibited by the subject, at least one of the following: a) using the data obtained from surface-of-section embedding techniques to determine an improvement in the base model within the domain of chaotic behaviour, by modifying at least one of the following: i) At least one equation; and, ii) At least one model value; and, b) using the data obtained from surface-of-section embedding techniques, to determine an improvement in the base model outside the domain of chaotic behaviour, by modifying at least one of the following: i) At least one equation; and, ii) At least one model value.

Typically the method includes, in the processor of the processing system: a) selecting a base model from a number of predetermined base models; and, b) modifying the base model to thereby simulate a condition within the subject.

Typically the base model is formed from at least one of: a) biological components; b) pharmacological components; c) pharmacodynamic components; and, d) pharmacokinetic components.

Typically the measured subject attribute is the subject status and the model value is a model output value indicative of the modelled subject status.

Typically the subject is at least one of a patient, an animal or an in vitro tissue culture.

Typically the treatment regime is indicative of: a volume of the at least one dose of the substance; a concentration of the at least one dose of the substance; a time at which the at least one dose of the substance is administered; and, a duration over which the at least one dose of the substance is administered.

Typically at least one of: the one or more state variables include an insulin attribute indicative of blood insulin levels; the one or more parameters include at least one of: a blood glucose concentration increase for a given dosage of glucose administered to the subject; a length of time for which blood glucose concentrations influence the pancreatic insulin secretion; an insulin release rate; and, a blood glucose concentration due to baseline liver glucose release; the one or more control variables include at least one dosage of glucose administered to the subject;

In one broad form, an aspect of the present invention seeks to provide apparatus for use in treating diabetes within a biological subject, the apparatus comprising a processing system having a processor for performing functions comprising: a) determining measured subject attributes of the biological subject, the measured subject attributes being periodically measured over a time period and wherein the measured subject attributes include a glucose attribute at least partially indicative of blood glucose levels; b) determining a base model including one or more equations including at least one non-linear ordinary differential equation or difference equation and wherein the model includes associated variables and parameters including: one or more state variables representing rapidly changing subject attributes, and including the glucose attribute; one or more parameters representing slowly changing or constant subject attributes, and including at least one of: a glucose disappearance rate constant; an insulin disappearance rate constant; a plasma glucose concentration increase for a given dosage of glucose administered to the subject; and, a plasma insulin concentration increase for a given dosage of insulin administered to the subject; and, one or more control variables representing attributes of a biological response of the subject that can be externally controlled using control inputs provided to the subject, and wherein at least one control input represents medication that can be provided to the subject, the one or more control variables including at least one dosage of insulin administered to the subject; c) calculating at least one model value using the base model, the at least one model value being a value of at least one of a variable, a control variable and a parameter; d) comparing the measured subject attributes and at least one corresponding model value to determine a difference between the measured subject attributes and the at least one corresponding model value, the different representing an accuracy of the base model, and the difference being determined by having the processor: i) derive a subject trajectory representing changes in the measured subject attributes over the time period; ii) calculate a model trajectory representing changes in the at least one corresponding model value over the time period; and, iii) perform the comparison by comparing the subject and model trajectories; e) modifying the base model in accordance with the determined difference, to improve said accuracy of the base model, the base the model being modified by modifying at least one of: i) at least one equation; and, ii) at least one model value; and, f) repeating steps c) to e) to iteratively modify the model to thereby generate a subject model representing the condition, the iterative modifying being performed until at least one of: i) the difference is below a predetermined threshold; ii) the difference asymptotically approaches an acceptable limit; and, iii) the difference is minimised; g) using the subject model to treat the condition within the subject by: using the model to derive a treatment regime including at least one dosage of a substance to be administered to the subject; and administering the at least one dose of the substance to the subject in accordance with the treatment regime to thereby treat the subject, and wherein the substance is at least one of insulin and glucose.

In one broad form, an aspect of the present invention seeks to provide a method of treating a condition within a biological subject, the method comprising, in a processing system having a processor and a memory: a) using the processing system to determine measured subject attributes of the biological subject, the measured subject attributes being measured over a time period; b) using the processing system to determine a base model including one or more equations including at least one non-linear ordinary differential equation or difference equation and wherein the model includes associated variables and parameters including: one or more state variables representing rapidly changing subject attributes; one or more parameters representing slowly changing or constant subject attributes; and, one or more control variables representing attributes of a biological response of the subject that can be externally controlled using control inputs provided to the subject, and wherein at least one control input represents medication that can be provided to the subject; c) using the processor of the processing system to calculate at least one model value using the base model, the at least one model value being a value of at least one of a state variable, a control variable and a parameter; d) using the processor of the processing system to compare the measured subject attributes and at least one corresponding model value to determine a difference between the measured subject attributes and the at least one corresponding model value, the difference representing an accuracy of the base model, and the difference being determined by having the processor: i) derive a subject trajectory representing changes in the measured subject attributes over the time period; ii) calculate a model trajectory representing changes in the at least one corresponding model value over the time period; and, iii) perform the comparison by comparing the subject and model trajectories; e) using the processor of the processing system to modify the base model in accordance with the determined difference, to improve said accuracy of the base model, the base model being modified by modifying at least one of: i) at least one equation; and, ii) at least one model value; f) using the processor of the processing system to repeat steps c) to e) to iteratively modify the base model to thereby generate a subject model representing the condition, the iterative modifying being performed until at least one of: i) the difference is below a predetermined threshold; ii) the difference asymptotically approaches an acceptable limit; and, iii) the difference is minimised; and wherein the iterative process includes: using external control inputs by administering medication to the subject to induce a perturbation or agitation of a status of the subject; and, determining at least one measured subject attribute whilst the perturbation or agitation is induced so that the model can be modified to simulate the application of the control inputs; and, g) using the subject model to treat a condition within the subject by: using the model to derive a medication regime; and administering medication to the subject in accordance with the medication regime to thereby treat the subject.

It will be appreciated that the broad forms of the invention may be used individually or in combination, and may be used in diagnosing the presence, absence or degree of medical conditions, treating conditions, as well as determining a heath status for a subject.

### BRIEF DESCRIPTION OF THE DRAWINGS

An example of the present invention will now be described with reference to the accompanying drawings, in which:

FIG. 1 is a flow chart of an example of a process for determining a model;

FIG. 2 is schematic diagram of an example of the functional elements used in determining a model;

FIG. 3 is a schematic diagram of an example of a processing system;

FIG. 4 is a flow chart of a specific example of a process for determining a model;

FIG. 5 is a schematic diagram of example solution trajectories;

FIG. 6 is a schematic diagram of a distributed architecture;

FIG. 7 is a schematic diagram of a generic pick-and-place industrial robot;

FIGS. 8A and 8B are sketches illustrating the concept of control using a Lyapunov function V(x);

FIG. 9 is a schematic diagram of an example model;

FIG. 10 is a graph of an example of a first insulin dosing strategy;

FIG. 11 is a graph of an example of expected and observed blood glucose concentrations under noiseless conditions using a relatively lax control and convergence regime for the first insulin dosing strategy;

FIG. 12 is a graph of an example of expected and observed plasma insulin concentration for the first insulin dosing strategy;

FIG. 13 is a graph of an example of expected and observed blood glucose concentrations under noiseless conditions using a tighter control and convergence regime for the first insulin dosing strategy;

FIG. 14 is a graph of an example of a second insulin dosing strategy;

FIG. 15 is a graph of an example of expected and observed blood glucose concentrations under noiseless conditions using a relatively lax control and convergence regime for the second insulin dosing strategy;

FIG. 16 is a graph of an example of expected and observed plasma insulin concentration for the second insulin dosing strategy.

### DETAILED DESCRIPTION OF THE PREFERRED EMBODIMENTS

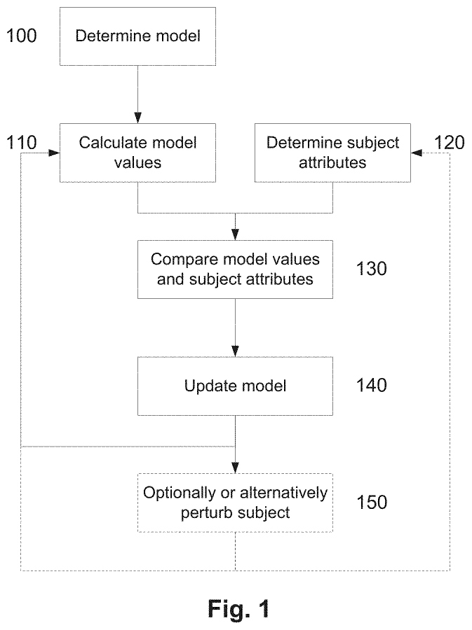

An example of a process for determining a subject model will now be described with reference to FIG. 1.

The subject model is intended to model the biological response of the subject. This allows the model to be used for example, to determine the health status, or the presence, absence or degree of one or more medical conditions. This also allows the biological effect of a condition to be modelled, allow derivation of treatment regimes or the like. The manner in which the model is derived will now be described in more detail.

For the purpose of the following examples, it is assumed that the subject is a human patient, but it will be appreciated that the techniques may be applied to any form of biological system including, but not limited to, patients, animals, in vitro tissue cultures, or the like. It will also be assumed that the process is being used to derive a model specific to a condition suffered by the subject, although it will be appreciated that this is not essential.

At step 100 a base subject model is determined. The model is typically in the form of a set of Ordinary Differential Equations (ODEs) or Difference Equations (DEs) that can be used to express basic responses of a subject. In this regard, the ODEs or DEs typically utilise a mixture of variables and parameters to represent the condition within the subject, including:
State variable values representing rapidly changing attributes;
Parameter values representing slowly changing or constant attributes; and,
Control variable values representing attributes of the condition that can be externally controlled.

The model can be determined in any one of a number of ways depending on the preferred implementation. For example, this can be achieved by selecting a predetermined model that is subject and/or condition specific. Alternatively, a model may be a default preliminary model, or can be selected from a range of different model components, depending on the condition or subject being modelled. The model could be derived manually by an operator.

At step 110 the model is used to calculate model values. The values can include any of the parameter or variable values, as well as a model output representing the overall expected status of the subject. The model values are typically calculated by applying one or more input values to the model (“model input values”) that represent the subject in some way. At the most basic level this can merely represent the progression of time, but can also take into account the subject environment, and any control inputs provided to the subject, such as medication, or the like.

Simultaneously, prior to, or in conjunction with this, the measurements indicative of subject attributes, such as the actual status of the relevant subject are determined. The subject attributes can be measured over a predetermined time period in advance of analysing the model, or alternatively can be measured in real time whilst the model is being generated.

In either case, at step 130, model values and subject attributes are compared to determine if the model is accurately represents the subject.

Thus, this typically involves measuring various physical parameters of the subject, such as biological markers, physical characteristics, or the like, and then comparing these to equivalent model values. This can therefore involve examining the overall status of the subject and comparing this to a model output. Alternatively, this can involve examining certain measurable attributes, such as concentrations of active substances and comparing this to a value derived from the model, which represents a theoretical concentration of the substance.

At step 140, the result of this comparison is used to modify or update the model to allow this to more accurately represent the subject and/or condition.

Typically this process will involve updating model parameter, state variable or control variable values, associated with the ODEs or DEs, although alternatively this may involve generating new, or replacement ODEs or DEs to update the model.

In any event, by iteratively performing this process it is possible to minimise or at least reduce variations between the model values and corresponding measured subject attributes so that the model more accurately represents the biological response of the subject. This can then be used to determine a health status and/or any medical conditions from which the subject is suffering.

As an additional option, it is possible to perturb or agitate the status of the subject through the use of external control inputs, such as providing medication to a patient, as shown at step 150. This allows further checks of the model to be performed, or generates further data. In this case, following application of the control inputs to the subject, subject attributes will be remeasured at step 120. The model is also modified to simulate the application of the control, and model values recalculated at step 110, before steps 130 and 140 are repeated.

It will be appreciated from this that the application of control inputs may be performed at any stage, including prior to any model value determination, as will be described in more detail below.

Once this is completed this allows the model to be used for any one of a number of different purposes. This can include, for example, using the model parameters to derive information regarding the subject that would not otherwise be easily or practicably measurable. Additionally the model can be used as a basis for a control program as described for example in co-pending International Patent Application No. WO2004027674.

Accordingly, the above described process allows a patient or other subject to be monitored, with the results of the monitoring being used to configure a model. This is achieved by comparing model predictions to the measured values, and then modifying the model to thereby minimise variations therebetween. Once the model is sufficiently accurate, this allows the model to be used in predicting the effects of medication regimes, or the like.

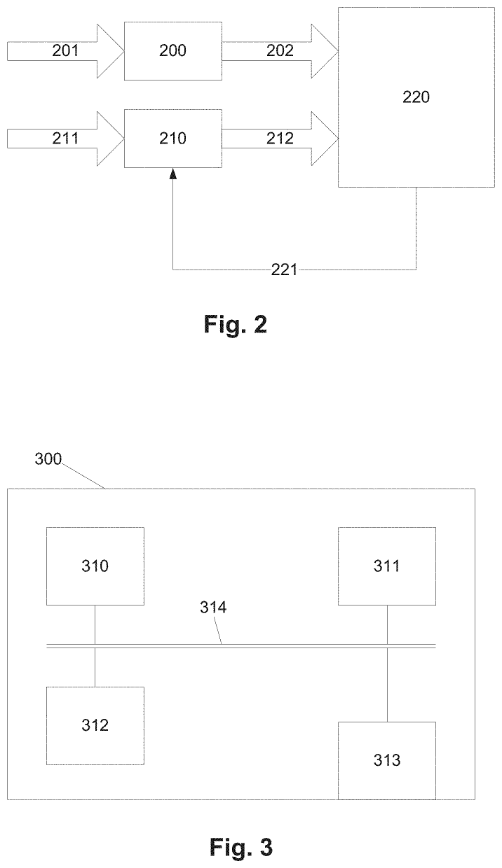

An example of the functional relationship of the subject and model is shown in more detail in FIG. 2.

In this example, a subject 200 has associated control inputs 201 in the form of medication or other external stimulus, with measured attributes being determined as an output at 202.

Similarly, the subject model 210 has corresponding model inputs 211 and model outputs 212. In this instance, the model inputs 211 typically correspond to control variable values representing the control inputs 201 applied to the subject 200. Similarly, the output 212 is typically formed from a combination of one or more state variable or parameter values obtained by applying the control variable values 211 to the model 210.

A control system is provided at 220 to analyse the measured attributes 202 and the model output 212 and provide feedback 221 to allow the model to be updated. This is typically achieved using some form of Model Reference Adaptive Control (MRAC) or related process of Identification, as will be described in more detail below.

A person skilled in the art will appreciate that aspects of the above outlined procedure may be performed manually. However, in order to achieve this it will be necessary for an individual to perform significantly complicated mathematics in order to analyse the measured subject attributes and calculate a suitable model. Accordingly, this process is typically performed at least in part utilising a processing system. In particular, the processing system is typically adapted to operate as the control system, as well as implementing the model 210. It will be appreciated from this that any suitable form of processing system may be used, and an example is shown in FIG. 3.

In this example the processing system 300 includes a processor 310, a memory 311, an input/output device 312, such as a keyboard, video display, or the like, and an external interface 313, interconnected via a bus 314.

In use the memory 311 will operate to store algorithms used in performing the comparison at step 130 and to allow update of model values at step 140. The memory 311 may also store parameter and variable values, as well as ODEs or DEs, associated with the model under consideration. Similarly, the processor 310 typically executes the stored algorithms to compare the subject's measured attributes to the model values and perform the necessary updates to the model.

Required inputs, such as the measured attributes, model details, and control inputs may be provided in any one of a number of manners. This can include receiving monitored or measured values from remote equipment via the external interface 313, or by having the information entered manually via the I/O device 312.

Thus for example, in the case of the measured attributes being derived from an MRI scan, the scan may be supplied directly to the processing system, which is then adapted to analyse the scan to extract the required information. Alternatively, a medical practitioner may be required to evaluate the scan to determine information such as the total brain cell mass therefrom, with this information then being submitted to the processing system.

Similarly, the models may be based on base models or model components that are input manually or retrieved from a store, such as the memory 311, a remote database, or the like.

It will therefore be appreciated that the computer system may be any form of computer system such as a desktop computer, laptop, PDA, or the like. However, as the level of processing required can be high, custom hardware configurations, such as a super computer or grid computing may be required.

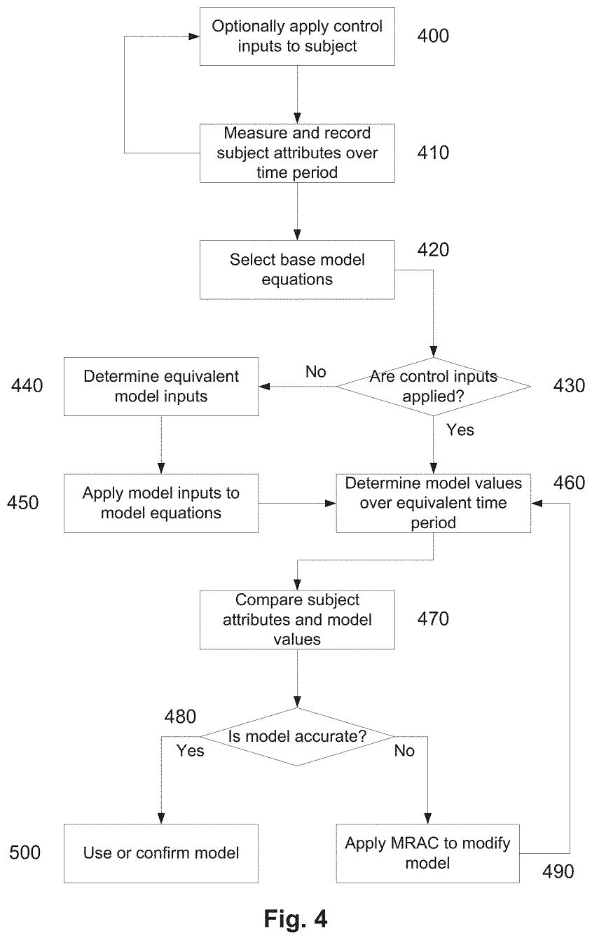

The process will now be described in more detail with respect to FIG. 4.

In this example, at step 400 control inputs are optionally applied to the subject, with the response of the subject, in the form of measured attributes, being determined and recorded over a time period at step 410.

Control inputs, where they exist may in any one of a number of forms, such as the introduction of a drug, or some other form of external stimulus. Control inputs may be set to null, or else actually implemented, depending on the circumstances.

The measured attributes can be in a range of forms, but typically is formed from a time-series of data representing one or a combination of:
the complete state of the system;
a fragment of the complete state of the system; and,
indirect measurements of one, some or all of the state variables.

It will be appreciated that the latter measurement can be achieved by measuring a related biological marker or response that is a function of the state variable of interest. Thus, for example, measuring the response of dopamine-responsive structures of a patient's eyes may serve as an indicator of dopamine concentration in the patient's cerebro-spinal fluid, when this dopamine concentration itself might not be easily or feasibly measured for practical reasons. Where relevant state variables cannot be measured for practical reasons, they are referred to as “hidden” variables.

This process can be repeated as often as required to generate a dataset for use in updating the model. Alternatively, or additionally, the steps 400 and 410 can be performed in conjunction with the remaining steps such that the model is updated in real time based on the current subject measurements, and this may depend on the manner in which the process is used. Thus for example, if this is used to model a patient suffering from a terminal condition, it may not be possible to collect a dataset in advance of the modelling due to time constraints.

At step 420 base model equations are selected. This may be performed by selecting from predetermined model components, such as physiological, pharmacokinetic or other biological model components, which are typically expressed as a system of ODEs. The model may be linearised, but this is typically of insufficient complexity to accurately model the condition within the subject, and accordingly, models are typically nonlinear.

The model will typically be in the form:

dz/dt=f(z,u,λ,t) (1)

where:
z is a state vector formed from the state variable values such that z∈Δ⊆ [symbol] ⁿ
Δ is a set of vectors of all possible state variable values
u is a control vector formed from control variable values such that u∈U⊆ [symbol] ^{p}
U is a set of vectors of all possible control variable values
λ is a parameter vector formed from the parameter values such that λ∈Λ⊆ [symbol] ^{q}
Λ is a set of vectors of all possible parameter values
t is time

It will be appreciated that in this instance, the model is made specific to the subject and the associated condition by selection of appropriate state variable and parameter values. To achieve this, the values may be initially seeded with default values, with the values being modified as described below, to allow the model to accurately represent the subject.

In the above example, the control vector u represents various external factors that can be used to influence the progression of the condition, such as the application of medication, or the like. The influence of these factors can be taken into account by examining the control inputs provided at step 400. Accordingly, at step 430, once the initial model has been selected, it is determined if control inputs are applied to the subject. If control inputs, such as medication, have been applied to the subject, then at step 440 equivalent model inputs are determined, and then applied to the model equations at step 450. This is typically achieved by modifying control variable values.

Once this is completed, or otherwise in the event that control inputs are not provided, the model is used to derive output in the form of one or more model values, such as state variable or parameter values. They are calculated over a time period equivalent to that over which the subject attributes were measured at step 460. Thus, for example, if model inputs are provided, these will be modified as required over the time period to represent the control inputs applied to the subject. Otherwise, if control inputs are not applied, then the model output is simply based on the progression over time with no inputs.

At step 470, the processing system 300 compares the subject attributes and model outputs over the time period and determines if the model is sufficiently accurate at step 480.

This can be achieved in a number of manners, but is typically achieved by determining if the difference or “error” between the measured attributes and the model values approach or fall below some acceptable limit or threshold. Thus, for example, this can involve examining the overall status of the subject and comparing this to an overall model output. Alternatively, the process can examine specific state variable and/or parameter values, and compare these to equivalent quantified biological attributes, to thereby determine if there is suitable convergence between the model and the subject's actual physical status.

Convergence is usually determined by mapping the time-series values of either the parameter values or state variables and equivalent biological attributes into state-space or phase-space, where they form trajectories. Thus, the ODEs or DEs forming the model are solved for the given time period to determine the change in values of the relevant parameters and/or state variables. Thus, once the equations have been determined, this allows the system equations to be used to generate solution trajectories φ, such that:

φ(z,u,λ,t)⊆ [symbol] ⁿ (2)

With the model representing the condition of the subject, the trajectories generated will represent a calculated route of progression of the condition within the subject, for the current model. By comparing this to measurements obtained from the subject, which represent the actual progression of the condition within the subject, this allows the accuracy of the model to be assessed.

In this example, the model and the subject's physical status are said to converge if the trajectories representing the state variables of the model and the biological attributes of the subject converge appropriately in the designated space.

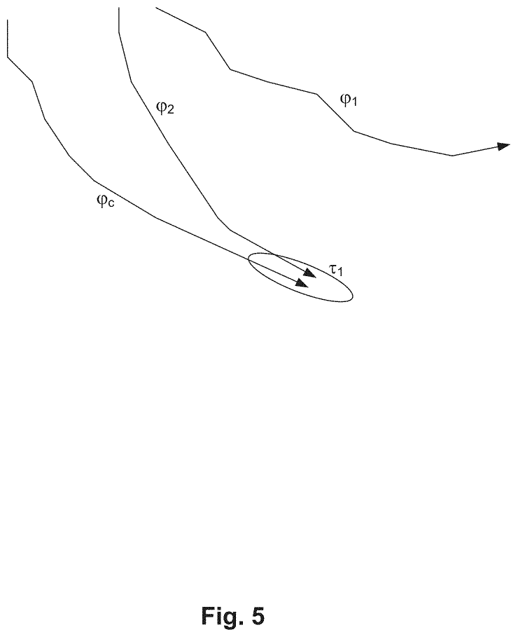

An example of this is shown in FIG. 5. In this example, the actual condition progression is represented by the trajectory φ_{c}. A first trajectory determined for the model progression is shown at φ₁, where it is clear that the condition and model diverge, whereas a second model trajectory is shown at φ₂, where it is clear that the condition and model converge as required.

It will be appreciated that convergence of the overall state, state variable or parameter values with the measured attributes may be employed individually, as distinct processes, or else in combination.

If the model does not converge then at step 490 MRAC or related methods are applied to modify the model parameter or variable values, or the equations. This can be achieved in a number of manners, and will depend on factors, including for example the nature of the model and whether this is linear or non-linear.

In a non-linear example, convergence of the model with the subject responsiveness can be achieved through the use of Lyapunov stability methods, which in turn may be ensured through use of suitable Lyapunov functions, denoted Vᵢ, and appropriate manipulation of the derivatives of these functions. The Lyapunov function can be generated as required, can be a specified analytical Lyapunov function, or can be determined by searching among derivatives of one or more candidate Lyapunov functions.

However this is achieved, the Lyapunov function, by forcing convergence or asymptotic convergence between trajectories, can then be used to generate estimates for any one or combination of model parameter, state variable or control variable values, that result in the best match between the model's predicted output and the subject's measured output. These values can then be incorporated into the model.

In one example, this is achieved by having the processing system 300 select a set τ₁ of desirable target points z_{τ} for model dz/dt=f(z, u, λ, t), based on the trajectory φ_{c} of the actual measured subject attributes. Medical histories, case studies or examination of the trajectories can also be used to define constraints on the vector of possible parameter values λ and the state variable values z, such that λ∈Λ_{τ} and z∈Δ, where Λ and Δ are bounded sets and Λ_{τ} is a compact set such that Λ_{τ}∈Λ.

Two Liapunov functions Vᵢ are then designed to allow improved parameter or state variable values to be determined. These are typically designed such that

Vᵢ:Δ×Λ→ [symbol] , (3)

where the operator symbol × in the above formula denotes a Cartesian product. In the case of V₁, this function is designed such that

τ₁⊆{z∈Δ: V₁(z,λ)<κ∀λ∈Λ_{τ}, for some specified κ>0}. (4)

The function V₂ will be designed to impose convergence between the model parameter estimates and the parameter values of the subject. The Lyapunov condition

\dot{V}ᵢ=∇Vᵢ•f<0 (5)

is then imposed on each function, where the operator • denotes a “dot product”. This simultaneously generates a medication regime that forces φ₂ and φ_{c} into τ₁, to converge while λ converges with the actual subject's parameter values.

A further variation is to use pattern-finding algorithms, or optimisation algorithms such as simulated annealing or genetic algorithms, to facilitate locating the best estimates for the values of model parameters in parameter-space, or to employ this process to optimise the relevant Lyapunov function and/or derivative for parameter identification. Similarly, pattern finding or optimisation algorithms can be used to assist in locating the best estimates for the values of hidden state variables in state-space, or employ this process to optimise the relevant Lyapunov function and/or derivative for reconstructing hidden state variables.

In addition or alternatively to this, it is also possible to use perturbations to the system by a fluctuating control input, to enhance or facilitate the identification process by pushing the system away from equilibrium conditions, as well as to address the presence of noise in the system.

For example, the model can also be adapted to take into account, or can be configured, using chaotic behaviour. The risk of mathematical chaos in a limited number of medical conditions, such as physical cardiac arrhythmia; is known. However the presence of mathematical chaos in clinical medication and in broader medical or biochemical applications is much wider than currently envisaged, for two reasons as outlined below.

Firstly, the majority of medication tasks in a clinical, other medical or biochemical context are, in mathematical terms, an exercise in forcing a dissipative or damped system. An example of this is using doses of medication, repeated regularly over time, to maintain the concentration of a ligand in an organ to a desired level or interval of levels, despite the ongoing presence of biological and physical processes, such as protein transport processes or enzyme-mediated reactions, that eliminate the ligand from the organ. Mathematically, the forcing of a dissipative system is known to be susceptible to the onset of chaotic behaviour, given appropriate parameter values;

Secondly, traditional pharmacokinetic formulations of drug uptake or absorption, distribution or transport, metabolism and elimination ignore a mathematical aspect that appears trivially obvious to biochemists: drug or ligand concentrations can never be negative. Consequently, a more accurate description of pharmacokinetic processes would include so-called “Heaviside” or “step” functions, mapping negative concentrations to zero. Incorporation of step functions in pharmacokinetic difference equations makes them non-invertible, which means that even low-dimension systems become more susceptible to chaos than indicated in usual models.

Reconstruction of phase-space information of a chaotic system, using delay coordinates and embedding, is a known process in advanced physics. Consider a system whose dynamics is described by a smooth (or piecewise smooth) low-dimensional set of ordinary differential equations,

dx(t)/dt=F(x(t)), for some function F.

The vector x(t) describes the state of the system, such that only one or more limited components of x(t) are able to be measured, or more generally, one or more scalar functions gᵢ(t) of the state of the system,

gᵢ(t)=Gᵢ(x(t)), for some scalar functions Gᵢ, where i=1, . . . , n, some n≥1, are able to be measured.

Then, using a surface-of-section map as described in the mathematical literature, e.g. E. Ott, Chaos in Dynamical Systems, Cambridge University Press 1993, it is possible to reconstruct information on the geometry of the attractor and the underlying dynamics of the system, including the function F, when the system is mathematically chaotic.

Accordingly, when the system, in this case the subject, is exhibiting chaotic behaviour, surface-of-section embedding techniques can be used to derive parameter values, and hence to refine the model. In particular, the data obtained from surface-of-section embedding techniques can be used to determine an improvement in the model by modifying either one of the equations used in the model, or one or more of the model values. This can be performed either in the domain of chaotic behaviour, or in the domain of non-chaotic behaviour.

The use of chaotic behaviour in this fashion may be required in a number of circumstances.

For example, the subject may be exhibiting mathematically chaotic behaviour when the process of forming the model is initially commenced.

Alternatively, in some circumstances, insufficient information may be obtainable purely from analysis of non-chaotic subject responses. In this case, by perturbing the subject, for example through the use of a suitable medication regime, mathematically chaotic behaviour can be induced within the subject.

It will be appreciated that analysis of subject response whilst undergoing mathematically chaotic behaviour may be used in addition to, or as an alternative to, the use of Lyapunov functions. For example, in some scenarios, use of Lyapunov functions alone will not allow sufficient refinement of the model, or allow sufficient parameters values to be determined, in which case, monitoring of the responsiveness in chaotic domains can be used to obtain or supplement required information.

Once regions of chaotic response have been determined, these can also be avoided in future, for example, through the use of a suitable medication regime, thereby assisting in subject treatment.

A further alternative is to construct the model from linear or linearisable systems of Ordinary Differential Equations (ODEs). In this instance, a linear error equation is formed, representing the difference between the desired state of the subject and the subject's actual state. The entire MRAC algorithm is then constructed around the problem of minimising this error.

In any event, once modifications have been determined, this allows the process to be repeated by returning to step 430 or step 460. This can be performed by comparing the model to the current dataset, as well as, or alternatively to comparing the model to a new dataset.

In any event, this process can be repeated until suitable convergence is achieved, at which point the model may be subsequently used at step 500. In particular, this allows the process to be performed iteratively until differences between the model and subject attributes asymptotically approach an acceptable limit or threshold.

It will be appreciated that the determined parameter values, state variable values, and/or equations may only be accurate over a short duration of time. Thus, as the condition progresses, it may be determined that the progression of the actual condition diverges from the trajectories predicted by the model. This may occur for a number of reasons.

For example, progression of the condition may cause an alteration in the model equations such that the model only accurately represents the condition for the current measured attributes. In this case, as the condition progresses, new equations, variable values, and/or parameter values, and hence new trajectories may need to be calculated to reflect the new subject condition. Additionally, the model parameters may be calculated based on limited information, such as a limited dataset, in which case it may be necessary to update the model as additional data becomes available.

Accordingly, the solution trajectories of the model can be repeatedly compared with the actual trajectory of the condition within the subject, allowing parameter, state variable, control variable values and/or equations to be recalculated, if convergence no longer holds.

The manner in which the model is used will depend on the particular circumstances. For example, the model can be used to determine the health status of a subject, for example by diagnosing the presence, absence or degree of conditions.

This can be achieved for example by deriving a model for the subject and then comparing the model to existing models to diagnose conditions. Additionally, or alternatively values of parameter values or state values can also be used in diagnosing conditions. Thus for example, deriving a state variable value representing dopamine levels can be used as an indicator as to the presence, absence or degree of conditions such as Parkinson's disease.

The model can also be used in treating patients, for example by deriving a medication regime, as described for example, in WO2004027674.

In addition to this, the model can be used to derive information regarding a subject that could not otherwise be actually or easily measured. Thus, for example, it is not always possible to determine certain physical or biological attributes of a subject. This can occur for example, if performing measurements is physically impossible, prohibitively expensive, or painful. In this instance, the model can be analysed to determine parameter or state variable values that correspond to the physical attribute of interest. Assuming that the model demonstrates suitable convergence with the subject, then this allows a theoretical value for the corresponding attribute to be derived.

In any event, by utilising feedback and in one example MRAC-based methods, this allows a model to be derived based on measured subject attributes. This is achieved by modifying the model in accordance with differences between measured subject attributes and corresponding model values. This can be performed iteratively to thereby minimise any variations, or at least reduce these to an acceptable level.

Thus, by ensuring the error between the measurements of output data from the medical system and the predicted output from the model asymptotically approach some acceptable limit (usually zero), this allows acceptable models to be mathematically derived.

In contrast to the original form of Model Reference Adaptive Control (MRAC) approach which updates the physical system to correspond to the predictive model, this technique emphasises updating the model until it is a suitable representation of the physical system.

### Architectures

It will be appreciated that the above method may be achieved in a number of different manners.

Thus, for example, a respective processing system 300 may be provided for each medical practitioner that is to use the system. This could be achieved by supplying respective applications software for a medical practitioner's computer system, or the like, for example on a transportable media, or via download. In this case, if additional models are required, these could be made available through program updates or the like, which again may be made available in a number of manners.

However, alternative architectures, such as distributed architectures, or the like, may also be implemented.

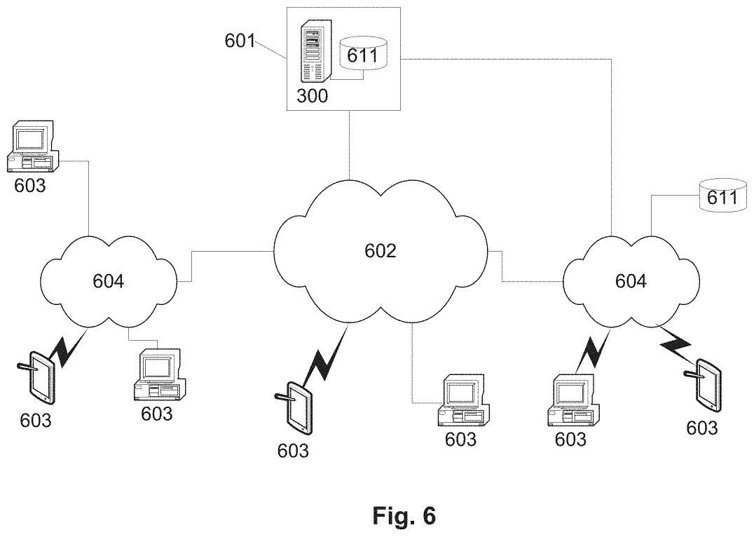

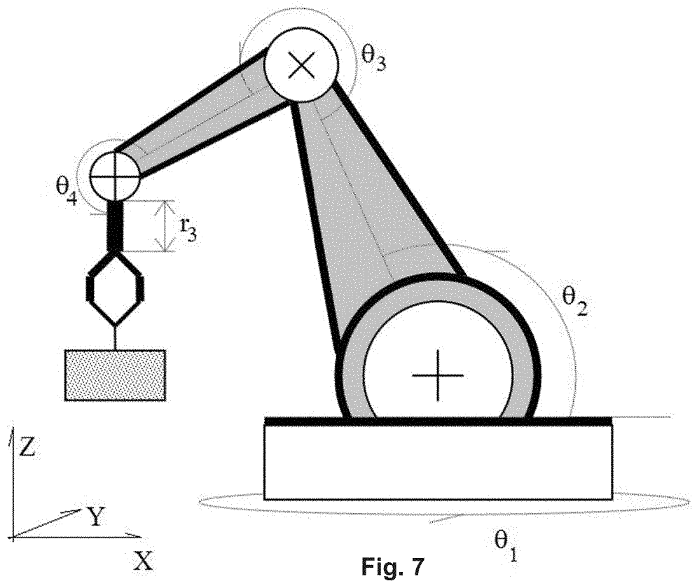

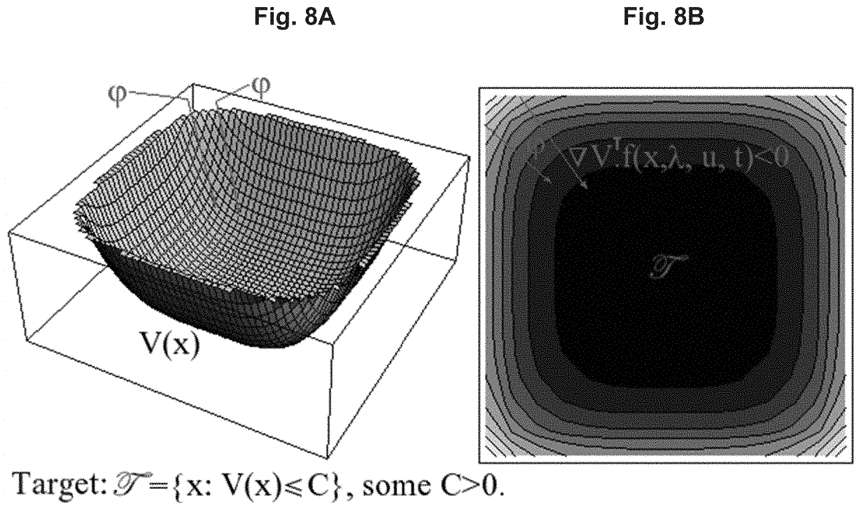

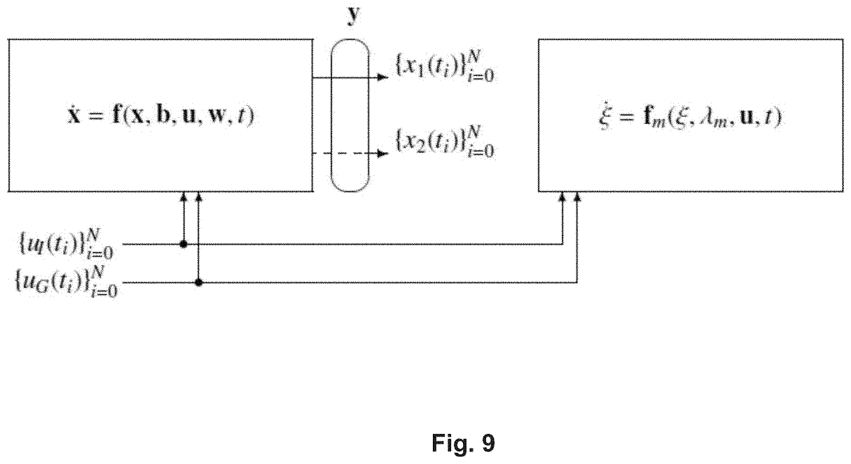

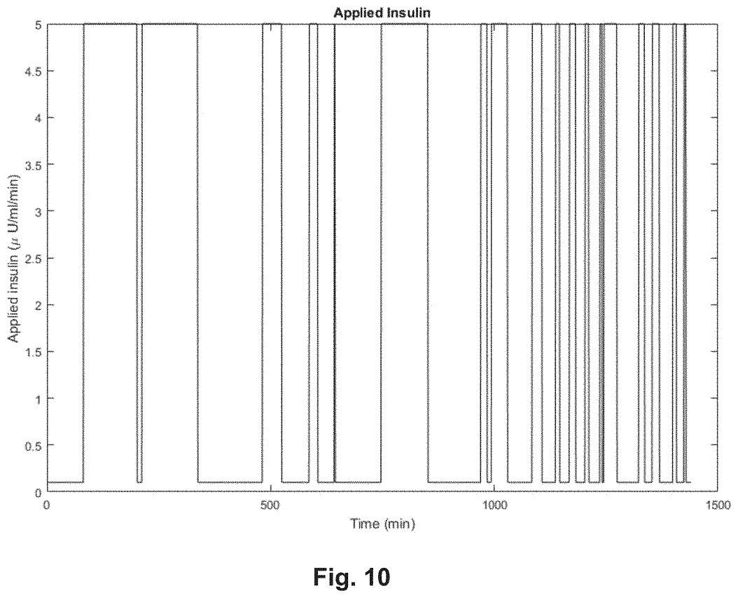

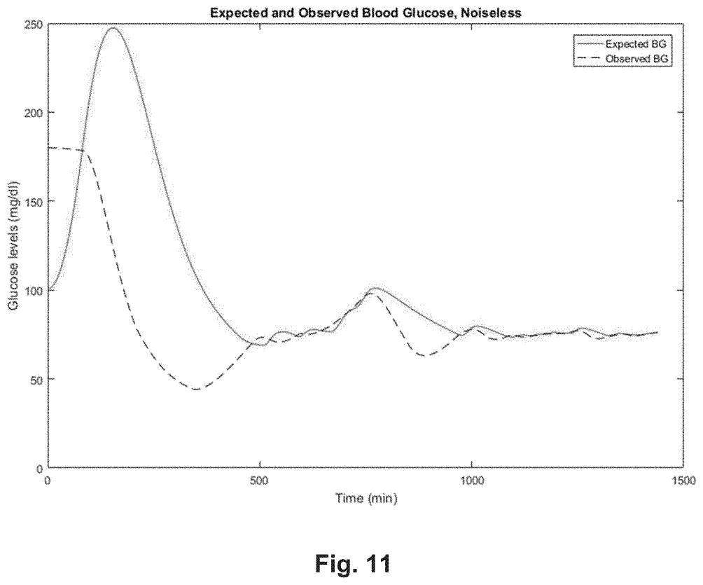

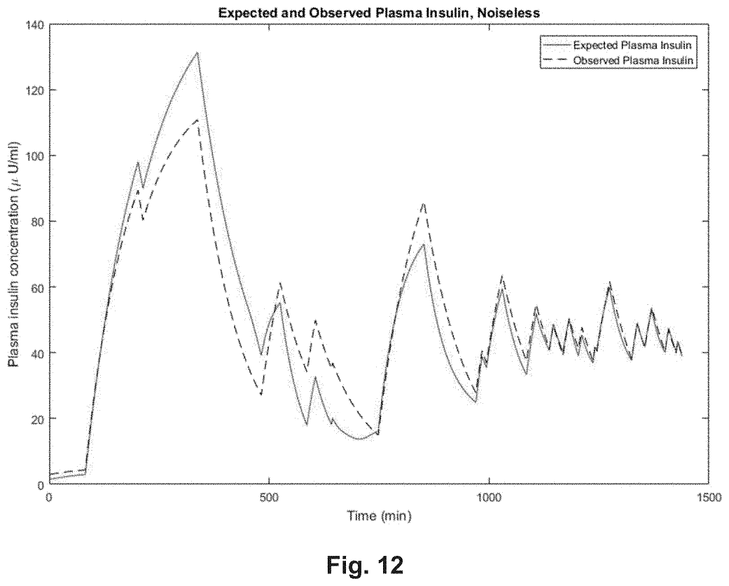

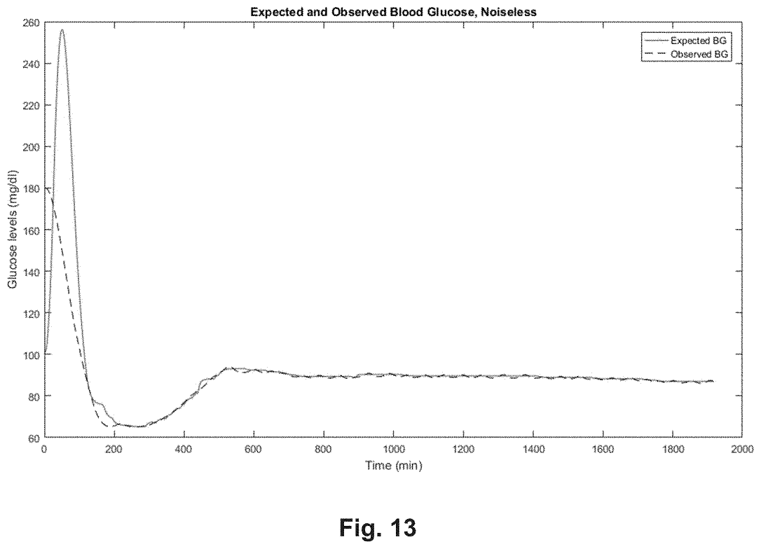

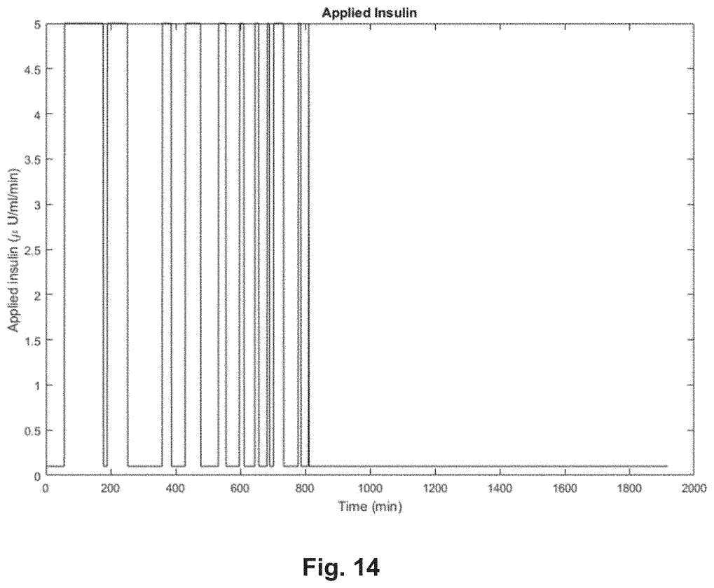

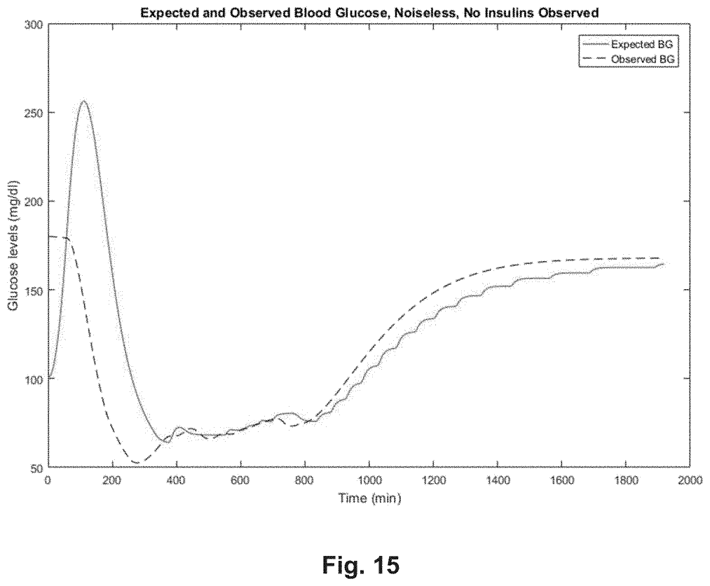

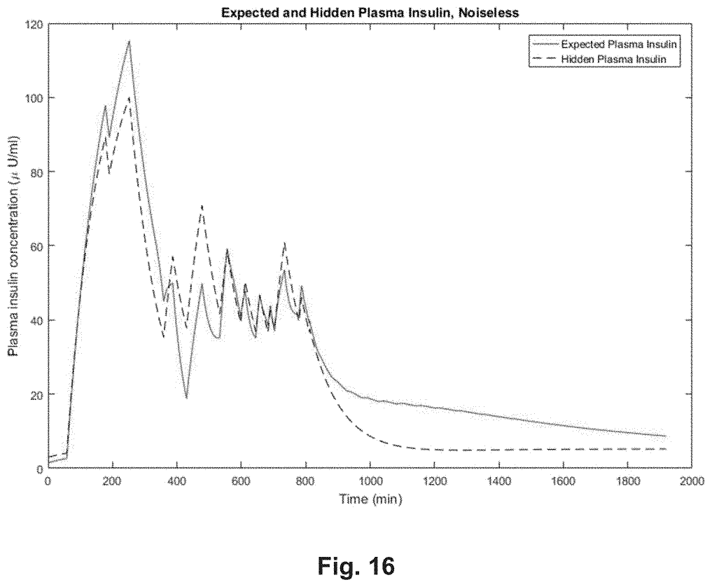

An example of this is shown in FIG. 6 in which the processing system 300 is coupled to a database 611, provided at a base station 601. The base station 601 is coupled to a number of end stations 603 via a communications network 602, such as the Internet, and/or via communications networks 604, such as local area networks (LANs). Thus it will be appreciated that the LANs 604 may form an internal network at a doctor's surgery, hospital, or other medical institution. This allows the medical practitioners to be situated at locations remote to the central base station 601.

In use the end stations 603 communicate with the processing system 300, and it will therefore be appreciated that the end stations 603 may be formed from any suitable processing system, such as a suitably programmed PC, Internet terminal, lap-top, hand-held PC, or the like, which is typically operating applications software to enable data transfer and in some cases web-browsing.

In this case, the data regarding the subject, such as the measured attribute values can be supplied to the processing system 300 via the end station 603, allowing the processing system 300 to perform the processing before returning a model to the end station 603.

In this case, it will be appreciated that access to the process may be controlled using a subscription system or the like, which requires the payment of a fee to access a web site hosting the process. This may be achieved using a password system or the like, as will be appreciated by persons skilled in the art.

Furthermore, information may be stored in the database 611, and this may be either the database 11 provided at the base station 601, the database 611 coupled to the LAN 604, or any other suitable database. This can also include measured subject attributes, determined models, base models, or components, example Lyapunov functions, or the like.

It will be appreciated that by analysing data collected for a number of subjects, this will allow more accurate models to be developed in an iterative process. Statistical analysis can also allow additional models to be developed, for example by analysing a range of age groups to create age-dependent models.

### Specific Example

A specific example in the context of treating diabetes will now be described.

In this example, the measured subject attributes include a glucose attribute at least partially indicative of blood glucose levels, which is measured continuously and/or periodically over a time period. The measurements could include blood glucose measurements obtained via a finger prick test or similar, or could include an alternative proxy for glucose measurements, such as measuring the interstitial fluid glucose (ISFG) using a continuous glucose monitor (CGM).

Additionally an insulin attribute indicative of blood insulin could also be measured. In general, this can only be performed in a clinic, so is less desirable, meaning the use of an insulin attribute is less likely, but could occur if needed and/or available.

The one or more state variables will include the glucose attribute and may also include the insulin attribute, should this be available.

As will be described in more detail below, additional state variables may be derived as part of the model, with these variables being artificial variables used to make the integro-differential equation of the dynamics work effectively, with values of these being estimated using the process described herein.

The one or more parameters typically include one or more of a glucose disappearance rate constant, an insulin disappearance rate constant, a plasma glucose concentration increase for a given dosage of glucose administered to the subject, or a plasma insulin concentration increase for a given dosage of insulin administered to the subject. Additionally, other parameters may be used, including but not limited to a length of time for which blood glucose concentrations influence the pancreatic insulin secretion, an insulin release rate and a blood glucose concentration due to baseline liver glucose release, or the like.

The one or more control variables typically include at least one dosage of a substance to be administered to the subject, with the substance being insulin and/or glucose, so that the resulting treatment regime includes the at least one dose of the substance. The treatment regime could be specified in terms of a volume of the at least one dosage of the substance, a concentration of the at least one dosage of the substance, a time at which the at least one dosage of the substance is administered and/or a duration over which the at least one dosage of the substance is administered. It will be appreciated that such a dosage could be delivered in medicinal form, in the case of insulin, but could also be administered as part of a food or beverage in the case of glucose.

For example, the treatment regime may require that the subject ingest a certain quantity of glucose, whether in the form of food or beverage, and then within a certain time period, ingest a defined amount of insulin, which could be delivered as a single intravenous injection and/or could be delivered progressively over a period of time.

A paper setting out the development and testing of a model will now be described. It will be noted that this approach uses pre-existing models, but applies the iterative approach described herein to more accurately determine subject specific variables and parameters, thereby rendering the model far more effective at developing treatment regimes that can control a subject's blood glucose levels, and keep these within desirable constraints.

In this paper a new subject-specific method for using nonlinear models of pharmacokinetics (PK) and pharmacodynamics (PD) and partial timeseries sensor data, to achieve clinical reconstruction of the patient parameters and real-time control of clinically important attributes of the patient is outlined, by modifying algorithms originally designed for the nonlinear model reference adaptive control (MRAC) of industrial robots, the Product-State-Space Technique (PSST). Although the PSST was originally designed to force the robot's system dynamics to track the dynamics of a known model with asymptotic convergence, they are applied here to biological or medical systems. Given that the convergence of model and system dynamics under the PSST does not have a fixed direction, it is possible to force the model to converge to the system dynamics. This has particular value in a medical context: if the structure of this model for the biological system is known with sufficient fidelity, and if partial time-series data of appropriate state variables are available at suitable frequencies, these algorithms can be used to achieve partial or complete identification of the parameters for that patient's biological system. Even under conditions where partial information is too incomplete to permit rigorous identification, the asymptotic convergence between model and system may be used to achieve asymptotic convergence of clinically-important variables with their model counterparts, such that nonlinear control of the model generates clinically useful outcomes.

Because the algorithms were built upon the principles of Lyapunov stability, they can be applied globally to systems with highly nonlinear dynamics, imposing control upon the trajectories of a system of nonlinear differential equations without requiring either an analytic solution or simplifying assumptions (e.g. linearisation). Such control can include forcing the complex dynamics of the biological system and a model to converge and remain close in phase space, enabling the traits of the system to be studied via the necessary modifications to the model required to sustain this convergence.

As a case study, the problem of controlling a subject-specific parametric model of insulin-glucose dynamics in type-1 diabetes is simulated under partial information. For clarity this is done using a variant of the well-known Intravenous Glucose Tolerance Test (IVGTT) for a “minimal” model of fasting diabetic insulin-glucose dynamics. As benchmarks, the somewhat artificial scenarios of sensors able to perform continuous 4 Hz monitoring of blood glucose and plasma insulin (scenario 1) or only blood glucose (scenario 2) are modelled. In each case successful model convergence and blood glucose control are demonstrated. Insulin is assumed introduced directly, rather than infused subcutaneously.

It can be appreciated that further modifications of these basic scenarios to add various more usual clinical characteristics—introduction of a further variable for interstitial fluid glucose (ISFG) monitored by a continuous glucose monitor (CGM) to replace the continuous blood glucose sensor assumption, constraints on the data reporting of the CGM device, introduction of insulin infusion instead of injection, increasing the complexity of the model, et cetera-will all alter the tracking performance of the proposed algorithm, which would then be re-optimised by re-designing the Lyapunov functions and consequent modification of the gradient descent algorithm.

It should also be apparent that this approach could be employed clinically for other medical conditions for which adequate PK/PD models are known and appropriate clinical sensors are available to generate time-series data during a medical intervention. The dosing regime of the drug would then be decided, not according to conventional medical dosages, but in real time by the algorithm.

### Algorithms for Individualised Closed-Loop Nonlinear Adaptive Control of a Parametric Model of Type-1 Diabetes, from Partial Time-Series Information

### Nigel Greenwood

This previously unpublished research paper was supported by the Queensland Government Department of Health under its Medical Devices Financial Incentive Program (MDFIP) in 2009. The author would also like to thank Dr Jenny Gunton for her suggestions and support.

### INTRODUCTION

The complexity of biological systems has been of growing interest in recent decades to medical researchers and mathematicians alike. If one describes dynamic biological processes as embodied by differential equations, and such equations are highly nonlinear and coupled, then there are implications for the attempted medical control of such processes.

In particular, modern control theory teaches that, given dynamic processes of sufficient complexity,
1. Such processes cannot be reliably controlled by using empirical observe-modify-observe methods (so-called “manual” methods) of the form traditionally employed in clinical medicine; and
2. They also cannot be reliably controlled through iterative application of such empirical feedback methods, starting from the“best” settings as ascertained from a population, to an individual whose parameter values are sufficiently far from average (where being “far” from the mean value depends on the structure of the system being studied).

Consequently, in the international literature increasingly sophisticated mathematical methods have been brought to bear on the task of medicating biological systems, to steer the state of the system to a desired state and keep it there. This task reduces into four sub-tasks, the first three of which are design, identification (or some other process of numerical construction) and control. The fourth sub-task relates directly to the complexity of biological systems and the approximate nature of models, and might best be described as adaptive modelling. In the context of analytical modelling, the scope of each is as follows:
1. Design of an appropriate basic mathematical model suitable for describing the system and its interactions with the external world, including “external” inputs such as medication, and “internal” dynamic processes, such as the system's pharmacokinetics (PK) and pharmacodynamics (PD), that define system behaviour of interest;
2. Conversion of this conceptual model into an equivalent numerical structure for quantitative prediction of system behaviour. Common methods include traditional population-based statistical estimation of parameter values and non-parametric Bayesian analysis. A third method involves estimation of each individual system's parameter values based on convergence between a model's behaviour and that of the actual system, when each is responding to a known control input; a process known in engineering parlance as identification;
3. Exploitation of this numerical model to generate a feedback (i.e. closed-loop) control law, enabling implementation of an appropriate medication strategy that chooses future control values based on the current dynamic state. In contrast with feedback control programs that are entirely designed offline before implementation, such a medication strategy would ideally be adaptive in the sense of [45]: steering the system to a desired state and keeping it there, meanwhile adjusting itself in real time to respond to broader intervening changes in the system (such as shifts in parameter values). Ideally, no additional constraints (such as simplifying or linearising assumptions) should be imposed upon the model by the method used to generate a control law;
4. Given the complexity underlying most biological systems, it is unlikely the initial model will represent a perfect description of the dynamics. Consequently the concept of adaptive control in the identification algorithms should be extensible to adaptive changes in the model structure, translating persistent discrepancies between actual and expected system behaviour, accreted over a sufficiently large number of observations, into structural enhancements of the model being used, improving the degree of stable convergence between expected and observed results.

Type-1 diabetes (T1D) is of particular interest as an example of the application of mathematical methods to medical control problems. Multiple models exist of various degrees of complexity, even the simplest of which (the Bergman “minimal” model, [2, 3]) is composed of nonlinearly coupled ordinary differential equations (ODEs). Glucose monitors and insulin pumps have been increasing in sophistication, but achieving an artificial pancreas that “closes the loop” for automated insulin control of glucose levels requires sophisticated software, to translate these improvements in sensors and infusion systems into a proportionate enhancement of therapy.

In this article, an appropriate set of algorithms is proposed for nonlinear identification, stable adaptive control and adaptive modelling, all occurring in real time. Built from Lyapunov stability methods, it takes a basic nonlinear model (in this case, of insulin control of glucose levels in fasting T1D) constructed from ODEs of state variables with constant parameters. This set of algorithms engages simultaneously in partial nonlinear identification, reconstructing the values of state variables and parameters from partial information obtained from time-series measurements, and closed-loop control, to steer the key variable of the system (blood glucose) successfully to a desired state using constrained control values.

These techniques can be extended to the task of revising the underlying model, constructing and implementing increasingly sophisticated models from medical data as it accrues.

### Model Reference Control in the Literature

As remarked previously, an accepted “minimal” mathematical model has existed since 1979, formulated by Bergman et al. from the Intra-Venous Glucose Tolerance Test (IVGTT). In a more recent mathematical study, De Gaetano and Arino [11] criticised the design of the Bergman model on stability grounds, reformulated it in the context of the IVGTT, and suggested an alternative low-dimension dynamical model of the glucose-insulin system. Both the reformulated version of the IVGTT “minimal” model and the alternative proposed in [11] are listed in Makroglou et al. [034], which gives a thorough overview of recent models of type-1 diabetes using different mathematical architectures, including the equation sets of eleven distinct models of greater complexity than the IVGTT “minimal” model.

If this information is to be used to assist actual stabilisation of blood glucose levels for diabetics, it raises two questions: which of these models is the best candidate to be employed, given limited data sets; and how is this model to be translated into practical medicine to improve therapeutic procedures?

The literature has demonstrated significant advantages in using mathematical methods for insulin-based control of glucose levels, over traditional “manual” control of insulin dose, both for diabetic subjects and otherwise-healthy subjects in intensive care (e.g. [39]).

But a key problem for the mathematical techniques employed in the literature is the nonlinearity of the biological system underlying the relationship between applied insulin and glucose levels in the blood and plasma, and the fact that not all of the relevant state variables are directly observable. This implies operational limitations and hence raises safety concerns when applied in a clinical context.

For linear systems a common analytic method is linear dual control. The system is controlled at all times, but control needs to be refined as soon as possible by improving knowledge of the underlying structures and parameters of the system dynamics, hence the “dual” nature of the method: control plus an increasingly accurate estimate of the numerical model. The basic form of dual control theory currently translated to a medical context is linear Model Reference Adaptive Control (MRAC).

This method is originally derived from a specific problem in engineering: for complicated machinery or electronic circuitry, the desired combination of identification and control processes is somewhat different from “true” identification. Rather than concentrating on reconstructing the state and parameter values for the most realistic model of a machine, the practical imperative has been to coerce that machine's behaviour into resembling that of a simplified model, which will follow a desired path through state space under a specified control program (hence “Model Reference”). If the dynamical system f is linear or can be approximated by a linear function, Luenberger ([32, 33]) pioneered a method enabling adaptive identification of unknown states of such a system. Krassovskii, [29], demonstrated an analogous method for adaptive identification of linear parameters. Modern linear MRAC is built on this framework.

Some of the strengths and weaknesses of these methods have been discussed in medical contexts by Palerm, [38], who explored their use both for type-1 diabetes and for haemodynamics. Palerm employed Direct Model Reference Adaptive Control Theory (DMRAC) and described limitations on these linear methods; in particular, commented that
1. Restrictive mathematical assumptions concerning the biological system must be made (this is an important problem with the method, and will be discussed in more detail shortly); and
2. The basic theory has no provision for handling constraints (imposed upper and lower bounds) on control commands. This is a critical issue for drug-infusion control, where constraints on drug delivery are needed for safety.

Further mathematical features were therefore designed and incorporated into the medical control architecture outlined in [38], to handle rate and saturation limiting in the medication input, and time delays within the system and model. These features were sophisticated, incorporating fuzzy logic as well as systems theory, and experimentally tested in computer simulations and animal experiments.

However, [38] concluded that this approach meant that
Practical controller design aspects appeared to be time-consuming and onerous, even during the animal trials;
Model accuracy was of paramount importance from the outset: flaws in the haemodynamic model used by [38] meant that its medication predictions were directly contradicted during the animal experiments;
More rigorous stability analysis was needed for the methods developed, and remained a open research topic.

Further criticisms can be made of linear dual control methods. Of these, the first two have been made in an engineering context in [45], while [12] has similarly laid out the constraints relevant to the third:
1. They are designed for linear or linearisable systems. But in both machines and organisms, linear examples are vastly outnumbered by their nonlinear counterparts. When used for nonlinear systems the Luenberger observer can only be applied locally to parts of state space via approximations (typically using Taylor series expansions), not globally over the entire space;
2. Where it can be used, the Luenberger method relies on an error equation,

e(t)=x(t)−ξ(t) (1)

where ξ(t) is the estimate to the actual (hidden) state vector x(t). The error term e(t) is forced asymptotically to zero over time,

$${lim}_{t \to ∞} ‖ e ( t ) ‖ \to 0 , \qquad ( 2 )$$

thus achieving ξ(t)→x(t) as t→∞. But forcing the estimate ξ(t) to approximate x(t) to within a specified accuracy within a specified time is more problematic; and
3. This error-equation approach is simply not applicable to highly-coupled nonlinear systems. Dorf, [12] briefly discusses the two necessary properties underlying (and limiting) linear control, namely superposition and homogeneity, both of which must apply before linear methods can be used. These properties fail in a nonlinear coupled system.
4. Furthermore, when translating techniques into medical therapies, some medical researchers appear to have lost sight of the original engineering purpose underpinning linear MRAC. The fundamental design of linear MRAC means that in a medical context, the process takes a model of a biological system, further approximates this into a locally linear form, then forms a control law on the basis of compelling the biological system to conform to the expected local behaviour of this linearised model. Although this is done for simple biomechanical applications such as the cardiac pacemaker, there are significant questions whether this is an appropriate strategy for an endocrine therapy, and the stability of such a strategy, concerns (for example) alluded to by [38]'s mention of restrictive assumptions, model accuracy and the need for stability analysis.

The use of linear dual control methods for controlling the glucose-insulin system has also been demonstrated in silico by Palumbo and De Gaetano [40], based on the alternative glucose-insulin model proposed in [11]. This alternative model retains the nonlinear coupled term of the Bergman “minimal” model; its authors acknowledge the problem of control laws based on linear approximations of a nonlinear model, but claim to have achieved instead an exact linearisation of the system via a coordinate transformation and feedback linearisation, to generate an insulin control law. This feedback linearisation appears to be analogous to an equivalent technique in robotics known as the method of computed torque (see e.g. Franklin, [16]). Unfortunately this method is difficult to scale to cope with additional system complexity, as it involves using part of the control effort to cancel the known nonlinear terms.

For safe algorithm-based control of medicated dissipative biological systems, a strong case can be made for explicit tracking and control of the underlying dynamics, with particular reference to subject-specific parameter values and changes in those parameter values. This is consistent with control engineering practice, which suggests the most robust (and hence safe) forms of control are achieved using explicit algorithmic control over well-mapped structures and evaluated parameters, provided the control algorithms can be made to scale to the likely complexity of the system to be controlled. This question of scalability is central to the problem of automating control processes for the artificial pancreas, because of its associated stability implications.

From the late 1970s to the early 1990s, an alternative mathematical basis for modelling and controlling highly nonlinear mechanical systems was pioneered by Skowronski, Leitmann and colleagues in the field of industrial robotics (see [6, 7, 8], [41], [44, 45, 46]). Known as the Product-State-Space Technique (PSST), it is a form of nonlinear dual control, exploiting Lyapunov stability theory to enable simultaneous identification and real-time adaptive control of extremely complex, nonlinear and coupled dynamical systems in motion—typical industrial robotics application would involve twelve- to sixteen-dimensional systems, with highly-nonlinear dynamics including uncertain dissipative effects such as damping.

Additional advantages of this approach are:
1. This approach was originally designed to achieve nonlinear MRAC, whereby the direction of convergence was to force the robot system (in engineering parlance, the “plant”) equations to mimic the behaviour of the reference model. But the use of Lyapunov functions means that the algorithms can be scaled to increasingly complex models; if these are sufficiently high-fidelity, convergence between model and plant dynamics can be imposed in the other direction, forcing the model to converge to plant behaviour via a nonlinear identifier, leading to “true” system identification.
2. The control law generated by a Lyapunov function V(x) can readily be implemented in real time, as it is simply a question of finding the steepest gradient descent down the slope ∇Vᵢt from the current state x(t) of the system. Depending on the design of the function, such a minimum may exist in analytical form, or it may be calculated numerically. The PSST extends this to real-time (or near-real time) adaptive control, with a second Lyapunov function enabling ongoing nonlinear identification processes to run in parallel.
3. Using appropriately-designed Lyapunov functions, supported by appropriate globally-valid numerical methods (e.g. genetic algorithms or similar), these processes of identification and control can be made globally valid over state space, instead of merely locally valid.
4. Consequently, given appropriate datasets, nonlinear identification and control of individual systems can be achieved globally using only partial time-series information, in the presence of noise and uncertainty. (The simulations shown here are noiseless for clarity, but the mathematical theory is presented to show how bounded noise terms would be incorporated.)
5. Explicit upper and lower bounds on medication control values or desired states of the system (as described by a target set in n-dimensional state space) can be specified prior to generating a control law, enabling the explicit imposition of safety constraints on system behaviour within the context of a particular model. (This is a feature translated from the original application in industrial robots, where a robot arm had to have spatial bounds imposed on its motion for safety in a crowded workspace.)
6. The region of controllability Δ_{c} for a specified Lyapunov function can be clearly determined. Within this region the control law is not only stable but, being derived from Lyapunov stability theory, typically imposes the strongest form of stability—asymptotically stable behaviour—on the system as its state is brought to the specified target set.
7. Given that no additional simplifications are imposed on the model in the formation of a Lyapunov function, any failures of control stability within Δ_{c} may indicate a discrepancy between the model and the underlying plant equations, and can be used to search for an improved set of model equations.

Consequently this new technique represents a promising approach for individualised therapeutic control of medicated conditions such as T1D, with the prospect of removing limitations currently imposed on the artificial pancreas project by inadequate identification and control methods.

### Formal Definitions in a Robotics Context

In translating an engineering technique into a medical application, it is useful to describe the technique first in its robotics context.

Expressed formally, the full information of a machine's dynamic state—e.g. the instantaneous internal positions and speeds of a robot's arms and manipulators, electric motors etc.—is embodied by its state vector, denoted x, where x=[x₁, x₂, . . . , xₙ]^{T}∈Δ⊂ [symbol] ⁿ for some n≥1, where Δ is the closed and bounded set of all physically possible state vectors.

The machine also has one or more parameters, describing values of attributes that affect the machine's functioning, e.g. oil viscosity, heat dissipation etc. These parameters may be constant, or they may change slowly over time, or may be modified in a limited way (e.g. changing oil temperature, viscosity and lubrication properties). The values for these various parameters are expressed as a vector, written λ, λ=[λ₁, λ₂, . . . , λₘ]^{T}∈Λ⊂ [symbol] ^{m}, some in m≥1, where Λ is the set of all possible parameter vectors.

Control variables exist, enabling partial manipulation of the system. The values of these controls are denoted by the control vector u=[u₁, . . . , uₚ]^{T}∈ [symbol] ⊂ [symbol] ^{p}, some p≥1, where [symbol] is the set of all available control vectors. There may also be noise or uncertainty in the system, designated by the vector w∈ [symbol] where [symbol] is the bounded set of all possible values of noise.

The general equation of motion of the machine, the so-called plant equation, system equation or system state equation, is then given by a vector-valued ODE with respect to time t, of the form:

\dot{x}=f(x,λ,u,w,t), (3)

for some function f, satisfying some initial condition x(t₀)=x⁰. The solution to this equation is the trajectory:

x(t)=φ(x⁰,t), (4)

This trajectory φ is a path drawn through η-dimensional state space, describing the instantaneous state of the machine for any given instant in time. Studies in the literature such as Filippov, [15], give sufficient conditions for trajectories to exist to a system of differential equations acting under a control program p. The family of such solution trajectories is denoted [symbol] (x⁰, t₀), further abbreviated to [symbol] (x⁰) for autonomous systems. The control program for which such trajectories exist is called admissible (see [45]).

The question is to calculate appropriate feedback control programs p for the controls u to steer the machine to a desired outcome, i.e. finding some p(x(t)) such that implementing

u(t)=p(x(t)) (5)

will force all solution trajectories φ(x⁰, t) to follow a desired path in state space for a specified period of time (perhaps indefinitely). If the dynamic structure f is known and the values for the machine's state x(t) and parameters λ can be measured, then control theory often enables a suitable closed-loop control program p(x(t)) to be calculated, steering the machine to perform desired behaviour.

Usually not all aspects of a machine's state or parameters can be measured: instead partial information forms an output vector y, where

y(t)=g(x,λ,w,t) (6)

for some function g. Under these conditions the task of identification, estimating the machine's unknown state and parameter values based on a time-series of (possibly noise-polluted) output measurements {y(tᵢ)} [symbol] ( [symbol] >>1), must be performed.

In this article, identification of a model will be demonstrated using Lyapunov functions, which are defined as follows:

Definition 1 (Lyapunov Function). Lyapunov (sometimes “Liapunov”) functions, named after the Russian mathematician A. M. Lyapunov (1857-1918), are defined to be any scalar-valued function

$$V ( x ) : {n}^{} \to$$

satisfying certain sufficiency conditions, but the form of which is otherwise arbitrary. These sufficiency conditions are to impose asymptotic stability and ensure the qualitative behaviour of a dynamical system within a particular region Δ₀⊂ [symbol] ⁿ without recourse to the calculation of solutions or approximation (e.g. linearisation about equilibria).

The usual form of sufficiency conditions used is exemplified in Franklin et al., whereby V(x) is required to satisfy:

$$\begin{matrix}V ( x ) is continuous , and has continuous derivatives with respect to all components of x : & 1. \\ V ( x ) > O , \text{?} ‖ x ‖ > 0 : & 2. \\ V ( 0 ) = 0 : & 3. \\ V = \text{?} \cdot f ( x , λ , u , \text{?} ) + \frac{\partial V}{\partial c} < 0 along system trajectories , and this condition reduces to \text{?} \cdot f ( x , λ , u , \text{?} ) < 0 for autonomous systems . & 4. \\ \text{?} \text{indicates text missing or illegible when filed} & \end{matrix}$$

Lyapunov functions are used for real-time or near-real time control of systems with complex dynamics, or systems requiring rapid and sophisticated decision-making in uncertain environments, e.g. multiple players in dynamic differential games: apart from previously-cited works, see also [17, 18], [22], [30], [47, 48], [49], [52], [56] for their use at different levels of machine decision-making.

There is no general algorithmic way to generate Lyapunov functions for a dynamical system; they are typically designed depending on the system's dynamics or its geometry. For example, a simple kinematic differential game contemplated in [17] or missile guidance application [52] had suitable functions constructed around Line of Sight (radial distance), whereas the complex dynamics of a robot arm or other complex mechanical system are typically controlled in [45, 46] using functions constructed from physical principles. For a particular dynamical system the choice of Lyapunov function is typically not unique and may be improved iteratively: in a medical context, to enlarge the region of controllability Δ_{c}, or to generate control strategies satisfying specified criteria (e.g. preferring “smooth” continuously-differentiable controls, typically corresponding with infusion, rather than discrete control values corresponding with injection), or to have Lyapunov target sets [symbol] corresponding with more clinically-useful targets.

### The Product-State-Space Technique

In this technique, a form of robot's system equation is written to show the effect of noise on motion: the so-called contingent equation,

\dot{x}∈{f(x,λ,u,w,t)|u∈p(x,λ,t),w∈ [symbol] }, (7)

with trajectory solutions φ(x⁰, t). The noise vector w contaminating the system behaviour is inherently unknown in value at any given instant, so the right-hand side of the equation shows a set of noisy elements. The model equation is written without noise:

\dot{ξ}=fₘ(ξ,λₘ,u,t), (8)

for some function fₘ, with trajectory solutions φₘ(ξ⁰, u, t).

Some of the robot's parameters can be adjusted, so the robot's overall parameter vector may be written λ(t) The model's parameter vector is taken to be constant, μm.

If the Lyapunov sufficiency conditions are imposed on a particular region Δ₀⊂ [symbol] ⁿ, the technique combines both these trajectories φ and φₘ into a single “product trajectory” steered from Δ₀ into a target state, wherein its two components will be dynamically kept balanced very close to equal (within a specified precision μ>0 for each of the state and parameter vectors), within finite time. The technique generates a combination of a control program p(x) (such that u(t)=p(x)) and an adaptive law fₐ for adjustable parts of the robot's parameter vector λ(t) such that

\dot{λ}=fₐ, (9)

so that the robot's manipulator arm ends up tracking the model's dynamics, obeying the user's commands to pick up and place objects as desired.

More formally (from [45]): the technique is based on the fact that is attained on a ∥x(t) −ξ(t)∥<μ “generalised diagonal” set within the space of product vectors [x, ξ]^{T}. Define the vector

Xₘ(t) [symbol] [x(t),ξ(t)]^{T}⊂Δ² (10)

where the set Δ² is defined to be

Δ² [symbol] Δ×Δ⊂ [symbol] ²ⁿ. (11)

Equations (7), (8) and (9) are then combined to form a product system incorporating the adaptive law. The structure of this product system is given by

F=[f,fₘ,fₐ]^{T}. (12)

Then define

α(t) [symbol] λ(t)−λₘ, (13)

which means that

\dot{α}(t)=\dot{λ}(t),t≥t₀. (14)

Then the product selector equation is

[\dot{X}ₘ(t),\dot{α}]^{T}=F(Xₘ,α,u,w,t) (15)

of the product system's contingent equation

[\dot{X}ₘ(t),\dot{α}]^{T}∈{F(Xₘ,α,u,w,t)|u∈p(Xₘ,α),w∈ [symbol] }. (16)

If Filippov's sufficient conditions for existence of solution trajectories are met ([15]), then the selector equation has the solution trajectories

$$φ ( {X}_{m}^{0} , {α}^{0} , . ) : -> {Δ}^{2} \times ⩓ , \qquad ( 17 )$$

where Xₘ⁰=Xₘ(t₀) and α⁰=α(t₀), generating at each [Xₘ⁰, α⁰]^{τ}∈Δ²×Λ the class of motions [symbol] (Xₘ⁰, α⁰).

Then the “diagonal” defined Δ²×Λ to be

[symbol]

{[Xₘ,α]^{T}⊂Δ²×Λ|x=ξ,α=0}, (18)

and its μ-neighbourhood is

[symbol]

_{μ}={[Xₘ,α]^{T}∈Δ²×Λ|∥x−ξ∥<μ,∥α∥<μ,}, (19)

for some nominated precision μ>0, where ∥·∥ denotes norms in [symbol] ²ⁿ and [symbol] ^{m} respectively.

Then [45] gives the following definitions and theorem (stated there as a “condition”):

Definition 2 (Tracking of Reference Model). The system (7) tracks the reference model (8) on Δ₀ with precision μ>0 if and only if there exists a control program p(·), an adaptive law f(·) and a time interval T_{μ}∈(0, ∞) such that Δ₀ is strongly positively invariant under p(·), and φ(Xₘ⁰, α⁰)∈ [symbol] (Xₘ⁰, α⁰) with [Xₘ⁰, α⁰]^{T}∈Δ₀²×Λ implies that

φ(Xₘ⁰,α⁰,t)∈ [symbol] _{μ},∀t>t₀+T_{η}. (20)

If T_{η} is a stipulated number, we speak of tracking after a given T_{η}.

Then, given the set Δ₀ where we want the tracking to occur, let [symbol] [∂(Δ₀²×Λ)] be an epsilon-neighbourhood of the boundary ∂(Δ₀²×Λ) of the region Δ₀²×Λ⊂ [symbol] ^{2n+m}, for some ∈>0. Define the semi-neighbourhood

[symbol]

= [symbol] [∂(Δ₀²×Λ)]∩Δ̅₀̅²̅×̅Λ̅ (21)

and the relative complement ( [symbol] _{μ}=(Δ₀²×Λ)/ [symbol] _{μ}.

Moreover, let some open set [symbol] ⊃ [symbol] be such that [symbol] ∩ [symbol] = [symbol] , and introduce two (Lyapunov) C⁻¹-functions

$${V}_{s} ( \cdot ) : -> and {V}_{u} ( \cdot ) : -> ,$$

such that

νₛ [symbol] Vₛ(Xₘ,α)|∀[Xₘ,α]^{T}∈∂(Δ₀²×Λ), (22)

ν_{μ}⁻ [symbol] infVₛ(Xₘ,α)|∀[Xₘ,α]^{T}∈∂(Δ₀²×Λ) [symbol] _{μ}∩ [symbol] (22)

ν_{μ}⁺ [symbol] supV_{μ}(Xₘ,α)|[Xₘ,α]^{T}∈∂(Δ₀²×Λ)∩ [symbol] (24)

Equation (22) requires constructing Vₛ(·) from a suitable ∂(Δ₀²×Λ) taken as a level curve of this function, or conversely by forming the boundaries of Δ₀ and Λ from level curves of a suitable Vₛ(·).

The strong controllability of a system is the capacity to control that system to a desired target state or outcome despite the adverse presence of noise or uncertainty. Such a desired outcome, e.g. successful collision by φ with a target set [symbol] , is known as a qualitative objective Q. More formally (from [45]):

Definition 3 (Strong Controllability). The system (7) is strongly controllable for some objective Q on Δ₀ at t₀ if and only if there is a program p(·) such that for each X∈Δ₀, motions φ(X⁰, t₀) exhibit Q for all φ(·)∈ [symbol] (X⁰, t₀). When p(·) and Q are independent of t₀, the system is strongly uniformly controllable for Q on Δ₀, and when Q is qualified by a stipulated time interval T_{Q} so is the controllability.

Then the main theorem for nonlinear identification is as follows, from Skowronski, [45]:

Theorem 4 (Strong Controllability for Model Tracking). The system (7) is strongly controllable on Δ₀ for tracking the model (8) according to Definition 2, if given Δ₀, Λ, μ, there exists a program p(·) and two (Lyapunov) C¹-functions Vs(·) and V_{μ}(·). as defined above, such that for all [Xₘ, α]^{T}∈Δ₀²×Λ we have:
1. Vₛ(Xₘ,α)≤νₛ,∀[Xₘ,α]^{T}∈ [symbol] ;
2. ∀u∈p(Xₘ,α),

∇Vₛ(Xₘ,α),F(Xₘ,α,u,w,t)<0,∀w∈ [symbol] ; (25)
3. 0≤V_{μ}(Xₘ,α)<ν_{μ}⁺,∀[Xₘ,α]^{T}∈ [symbol] ;
4. V_{μ}(Xₘ,α)≤ν_{μ}⁻,[Xₘ,α]^{T}∈ [symbol] ∩ [symbol] ;
5. ∀u∈p(Xₘ,α)∃c(∥[Xₘ,α]^{T}∥)>0, continuously increasing such that

∇V_{μ}(Xₘ,α),F(Xₘ,α,u,w,t)≤c(∥[Xₘ,α]^{T}∥),∀w∈ [symbol] . (26)

Condition (25) imposes asymptotic stability and strong controllability on the system to reach some target state, while condition (26) is used to force identification of the system despite the presence of noise or uncertainty.

As remarked in [45], this enables tracking within finite time and indeed enables the user to stipulate the period of time T, after which tracking is expected to exist. Defining

c⁻ [symbol] infc(∥[Xₘ,α]^{T}∥)|[Xₘ,α]^{T}∈Δ₀²×Λ, (27)

then the value for c>0 can be determined from the relationship

$${T}_{μ} = \frac{{v}_{μ}^{+}}{{c}^{-}} , \qquad ( 28 )$$

Transforming [4] into

$$∇ {V}_{μ} ( {X}_{m} , α ) \cdot F ( {X}_{m} , α , u , w , t ) \leq - \frac{{v}_{μ}^{+}}{{T}_{μ}} , \\ ∀ w ∈ . \qquad ( 29 )$$

This technique was originally developed to get a poorly-known complex system (the robot arm) to converge upon the dynamics and behaviour of a model. In the context of medical identification, the direction of this convergence in condition (26) is now reversed: the model is forced to converge on the dynamics of the system through tracking the trajectory while complying with a specified dynamical structure. This means the adaptive law (9) is re-cast for “true” identification: if the actual system has fixed parameters, equation (13) becomes

α(t) [symbol] λ−λₘ(t), (30)

which means that

\dot{α}(t)=−\dot{λ}ₘ(t),t≥t₀. (31)

and the adaptive aspect of control is manifested by the algorithms re-assessing the model's parameter values. This requires a fast global numerical search algorithm across the parameter set Λ, performed here using genetic algorithms.

Before this is demonstrated, an appropriate model for the glucose-insulin system must be chosen.

### A Model for Type-1 Diabetes

In 2000 a critique by De Gaetano and Arino, [11], was published of the Bergman minimal model and the associated two-stage approach to extracting a model from IVGTT data. In particular [11] made the following points:
In order to study glucose-insulin homeostasis as a single dynamical system, a globally unified model and a “single pass” method of reconstructing associated model parameters is highly desirable on both theoretical and practical grounds, as the glucose-insulin system is an integrated physiological dynamical system and the two-stage process effectively omits an internal coherency check;
Coupling the three minimal model equations leads to demonstrable structural and stability problems;
An alternative low-dimension model is needed to embody the glucose-insulin system, formulated in a more general way than being necessarily associated with the IVGTT experiment.

Consequently [11] proposes such an alternative model and offers both theoretical and experimental justification for it, using human data. This proposed model, which retains the key features of the Bergman model while resolving its associated structural and stability issues, is:

$$\begin{matrix}\frac{dG ( t )}{dt} = - {b}_{1} G ( t ) - {b}_{4} I ( t ) G ( t ) + {b}_{7} \\ \frac{dI ( t )}{dt} = - {b}_{2} I ( t ) + \frac{{b}_{6}}{{b}_{5}} {\int }_{t - {b}_{5}}^{t} G ( s ) ds\end{matrix} \} \qquad ( 32 )$$

with the associated initial conditions for an IVGTT being

$$\begin{matrix}G ( t ) ≡ {G}_{b} , ∀ t ∈ [ - {b}_{5} , 0 ) \\ G ( 0 ) = {G}_{b} + {b}_{0} , \\ I ( 0 ) = {I}_{b} + {b}_{3} {b}_{0} .\end{matrix} \} \qquad ( 33 )$$

In [11], insulin units for the coefficients in (32) are given in picomoles/litre (pM), where the authors state a conversion factor of 1 μU/ml=7 pM; however, using that conversion factor the coefficients in this article have had their units translated back into μU/ml, to ensure consistency with the subsequent paper by Palumbo and De Gaetano, [40]. The coefficients are then:

| | |
|---|---|
| t | [min] is time; |
| G | [mg/dl] is the glucose plasma concentration; |
| G_{b} | [mg/dl] is the basal (preinjection) plasma glucose concentration; |
| I | [μU/ml] is the insulin plasma concentration; |
| I_{b} | [μU/ml] is the basal (preinjection) insulin plasma concentration; |
| b₀ | [mg/dl] is the theoretical increase in plasma concentration over the basal |
| | level after instantaneous administration and redistribution of the IV glucose |
| | bolus; |
| b₁ | [min⁻¹] is the spontaneous glucose first order disappearance rate |
| | constant; |
| b₂ | [min⁻¹] is the apparent first-order disappearance rate constant for |
| | insulin; |
| b₃ | [(μU/ml)(mg/dl)⁻¹] is the first-phase insulin concentration increase per |
| | (mg/dl] increase in the concentration of glucose at time zero due to the injected |
| | bolus; |
| b₄ | [[μU/ml)⁻¹min⁻¹] is the constant amount of insulin-dependent glucose |
| | disappearance rate constant per unit plasma insulin concentration; |
| b₅ | [min] is the length of the past period whose plasma glucose |
| | concemirations influence the current pancreatic insulin secretion; |
| b₆ | [[μU/ml)(mg/dl)⁻¹min⁻¹] is the constant amount of second-phase insulin |
| | release rate per (mg/dl) of average plasma glucose concentration throughout the |
| | previous b5 minutes; |
| b₇ | [(mg/dl) min⁻¹] is the constant increase in plasma glucose concentration |
| | due to constant baseline liver glucose release. |

A drawback of the model proposed by [11], as presented in equations (32), is that these are integro-differential equations and hence difficult to translate into a control law. Consequently a subsequent paper, [40], first re-casts the integro-differential equations into a slightly more tractable form:

$$\begin{matrix}\frac{dG ( t )}{dt} = - {b}_{1} G ( t ) - {b}_{4} I ( t ) G ( t ) + {b}_{7} \\ \frac{dI ( t )}{dt} = - {b}_{2} I ( t ) + {b}_{6} {\int }_{0}^{∞} ω ( s ) G ( t - s ) ds\end{matrix} \} \qquad ( 34 )$$

where b₆ is re-dimensioned and the kernel ω(s) in the insulin dynamics is defined such that

∫₀^{∞}ω(s)ds=1,∫₀^{∞}sω(s)ds=T<∞, (35)

and T represents the average time delay. Palumbo and De Gaetano assert that ω(s) characterises the choice of the glucose-insulin model, and that all of the models described by (34) and (35) provide unique, positive bounded solutions and admit a unique, locally asymptotically stable equilibrium point given by the basal glycaemia and insulinaemia (G_{b}, I_{b}), with global asymptotic stability ensured if the average time delay T is sufficiently small.

The IVGTT initial conditions are then rewritten as

$$\begin{matrix}G ( t ) ≡ {G}_{b} ∀ t ∈ ( - ∞ , 0 ) \\ I ( t ) ≡ {I}_{b} ∀ t ∈ ( - ∞ , 0 ) \\ G ( 0 ) = {G}_{b} + {b}_{0} \\ I ( 0 ) = {I}_{b} + {b}_{3} {b}_{0}\end{matrix} \} \qquad ( 36 )$$

And [40] chooses ω(s) to satisfy

ω(s)=γ²se^{-ys},γ>0, (37)

so that

∫₀^{∞}ω(s)G(t−s)ds=∫₀^{∞}γ²se^{-ys}G(t−s)ds=∫_{-∞}^{t}γ²(t−τ)e^{-γ(}t−γ)G(τ)dτ. (38)

Then [40] defines the following state variables:

$$\begin{matrix}{x}_{1} ( t ) = G ( t ) , \\ {x}_{2} ( t ) = I ( t ) , \\ {x}_{3} ( t ) = {\int }_{- ∞}^{t} {γ}^{2} ( t - τ ) {e}^{- γ ( t - τ )} G ( τ ) d τ , \\ {x}_{4} ( t ) = {\int }_{- ∞}^{t} {e}^{- γ ( t - τ )} G ( τ ) d τ .\end{matrix} \} \qquad ( 39 )$$

which, using the linear chain trick, is then transformed into a four-dimensional model of the glucose-insulin system constructed from ODEs:

$$\begin{matrix}{\dot{x}}_{1} = - {b}_{1} {x}_{1} ( t ) - {b}_{4} {x}_{1} ( t ) {x}_{2} ( t ) + {b}_{7} + {u}_{G} ( t ) , \\ {\dot{x}}_{2} = - {b}_{2} {x}_{2} ( t ) + {b}_{6} {x}_{3} ( t ) + {u}_{l} ( t ) , \\ {\dot{x}}_{3} = - {γ x}_{3} ( t ) + {γ}^{2} {x}_{4} ( t ) , \\ {\dot{x}}_{4} = {x}_{1} ( t ) - {γ x}_{4} ( t ) .\end{matrix} \} \qquad ( 40 )$$

where the insulin and glucose control inputs (when applied) are denoted μ_{I}(t) and μ_{G}(t) respectively. Note the coupled nonlinearity x₁(t) x₂(t) in \dot{x}₁. The associated initial conditions for an IVGTT are then (from (36)),

$$\begin{matrix}{x}_{1} ( {t}_{0} ) = {G}_{b} + {b}_{0} , \\ {x}_{2} ( {t}_{0} ) = {I}_{b} + {b}_{3} {b}_{0} , \\ {x}_{3} ( {t}_{0} ) = {G}_{b} , and \\ {x}_{4} ( {t}_{0} ) = \frac{{G}_{b}}{γ} .\end{matrix} \} \qquad ( 41 )$$

These basal values G_{b} and I_{b} can themselves be determined from parameter values using the equilibrium conditions for (40), whereby

$$\begin{matrix}{G}_{b} = \frac{{b}_{2}}{2 {b}_{4} {b}_{6}} ( - {b}_{1} + \sqrt{{b}_{1}^{2} + 4 \frac{{b}_{4} {b}_{6} {b}_{7}}{{b}_{2}}} ) , \\ {I}_{b} = \frac{{b}_{6}}{{b}_{2}} {G}_{b} .\end{matrix} \} \qquad ( 42 )$$

Typical values for these parameter values are given in [40] for healthy, type-1 diabetic and type-2 diabetic subjects, reproduced in Table 1. (This data is internally redundant, enabling the validity of (42) to be reconfirmed.)

TABLE 1
Typical healthy and diabetic parameter values for the Palumbo-De Gaetano model

| Parameter | Healthy | Type-1 | Type-2 | Units |
|---|---|---|---|---|
| G_{b} | 79 | 180 | 150 | mg/dl |
| I_{b} | 62.5 | 3 | 40 | μU/ml |
| b₁ | 0.018063 | 0.018063 | 0.018063 | min⁻¹ |
| b₂ | 0.041342 | 0.041342 | 0.041342 | min⁻¹ |
| b₄ | 1. 10⁻⁵ | 6.457 10⁻⁴ | 1.484 10⁻⁴ | (μU/ml)⁻¹min⁻¹ |
| b₆ | 0.032707 | 6.890 10⁻⁴ | 0.011 | (μU/ml)(mg/dl)⁻¹min⁻¹ |
| b₇ | 1.4763 | 3.6 | 3.6 | (mg/dl)min⁻¹ |
| γ | 0.1022 | 0.1022 | 0.1022 | min⁻¹ |

This Palumbo-De Gaetano model is a unified global model of the glucose-insulin system, and this is suitable for demonstrating the medical form of the PSST with its adaptive law reversed for simultaneous partial “true” identification and adaptive control.

### Real-Time Reconstruction and Control of Fasting T1D Under Partial Information

Two scenarios of real-time adaptive control using the PSST were simulated using the equations of (40) to represent the diabetic glucose-insulin system, showing how this technique could be employed in real time under conditions of partial information.

To be consistent with the notation of (40), the vector of “true” parameter values, generically λ, will be denoted b [symbol] [b₁, b₂, b₃, b₄, b₆, b₇, γ]^{T}∈ [symbol] ⁶. The parameters b₃ and b₅ are not used in this model and are set to be null. Then the system that generated the diabetic data becomes

\dot{x}=f(x,b,u,w,t), (43)

where w=Q is assumed for clarity (system dynamics are noiseless).

For each simulation a value for the vector b was chosen, but this was hidden from the identifier, with the system generating data as a “black box”. The ODEs of (40) were converted to difference equations (DEs), with a time increment of 0.25 seconds. All sensors were assumed to operate at frequency 4 Hz.

Unlike the conventional IVGTT, there was no initial glucose bolus or subsequent introduction of glucose, so {μ_{G}(tᵢ)}ᵢ₌₀^{N} we wished to establish partial identification and control with insulin-only inputs, simulating a plausible clinical dual control of a subject under fasting conditions.

In all simulations, time-series measurements {y₁(tᵢ)}ᵢ₌₀^{N} of glucose concentration in plasma {x₁(tᵢ)}ᵢ₌₀^{N} were generated and made available in the output to the identifier, where in

y₁(tᵢ)=x₁(tᵢ)+w₁(tᵢ) (44)

where w₁(tᵢ) denotes noise associated with measurement (here null).

Two scenarios were simulated:
1. In the simulations of Scenario 1, time-series measurements {y₂(tᵢ)}ᵢ₌₀^{N} of insulin concentration in plasma {x₂(tᵢ)}ᵢ₌₀^{N} were also made available in the output, and hence similarly

y₂(tᵢ)=x₂(tᵢ)+w₂(tᵢ). (45)

where w₂(tᵢ) denotes sensor noise (here null).
2. In the simulations of Scenario 2, insulin concentrations were assumed unobservable, and hence {y₂(tᵢ)} had to be reconstructed from y₁(t) and a numerical approximation of \dot{y}(t).

State variables x₃(t) and x₄(t) were assumed always unobservable. No parameters could be measured directly, but lay within a known hypercube A specified in the table below:

TABLE 2
Table of Parameter intervals for the system, forming hypercube Λ

| Model Parameter | Interval for search |
|---|---|
| λm1 | [0.01445, 0.021676] |
| λm2 | [0.033074, 0.049610] |
| λm4 | [1.0 10−5, 7.0 10−4] |
| λm6 | [5.0 10−4, 5.0 10−3] |
| λm7 | [1.46, 3.6] |
| λm8 | [0.08176, |

Given the partial nature of this information, the notation for the Lyapunov functions in Theorem 4, expressed there in a general format, must be re-written for clarity and practical implementation. As much of x(t) and all of b was never visible, the functions Vₛ and Vᵤ are better denoted as Vₛ(y(tᵢ), ξ(tᵢ), λₘ(tᵢ) and V_{μ}(y(t), ξ(t), λₘ(t) respectively, and constructed accordingly.

The insulin control signal {μ_{I}(tᵢ)}ᵢ₌₀^{N} was generated at each point in time by a gradient descent algorithm applied to Vₛ(y(tᵢ), ξ(tᵢ), λₘ(tᵢ)), where Vₛ was designed as a positive-definite function, the dominant term of which was (x₁(tᵢ)−C₁)² for some suitable central value C₁ (80 mg/dl in Scenario 1 below, and 150 mg/dl in Scenario 2), such that (25) is satisfied. Then the noiseless version of (25) produces:

$${u}_{l} ( {t}_{i} ) = arg {min}_{u ∈ \vert {u}_{l}^{-} , {u}_{l}^{-} \vert} ∇ {V}_{s} ( y ( {t}_{i} ) , ξ ( {t}_{i} ) , {λ}_{m} ( {t}_{i} ) ) \cdot F ( y ( {t}_{i} ) , ξ ( {t}_{i} ) , {λ}_{m} ( {t}_{i} ) , u ( {t}_{i} ) , {t}_{i} ) , i = 1 , 2 , … \qquad ( 46 )$$

Simultaneously the candidate model parameter vector λₘ(t) was being modified at each point in time by a gradient descent process consistent with (26), so that at each new time an incremental improvement is made,

λₘ(tᵢ₊₁)=λₘ(tᵢ)+Δλₘ(tᵢ), (47)

where the particular increment Δλₘ(tᵢ) was chosen out of a set of candidate incremental changes Δλₘ∈ [symbol] at that moment, based on the particular gradient descent algorithm being used (e.g. fixed-step or variable-step descent),

$${Δλ}_{m} ( {t}_{i} ) = {V}_{μ} ( y ( {t}_{i} ) , ξ ( {t}_{i} ) , {λ}_{m} ( {t}_{i} ) ) \cdot F ( y ( {t}_{i} ) , ξ ( {t}_{i} ) , {λ}_{m} ( {t}_{i} ) , u ( {t}_{i} ) , {t}_{i} ) , i = 1 , 2 , … \qquad ( 48 )$$

Here V_{μ} was again a positive-definite function designed around the term μ₁(y₁(tᵢ)−ξ₁(tᵢ))²+μ₂(y₂(tᵢ)−ξ₂(tᵢ))², with weights μ₁>0, μ₂>0.

Numerical simulations were begun with all components of Δλₘ(t₀) taken at their minimum values in the hypercube, to ensure an initially large error, then to demonstrate the adaptive control. Depending on the design choice for Vₛ, the medication strategies generated could be continuous or pulsatile; in the present case pulsatile control values for μ_{I}(tᵢ) were regarded as acceptable.

The results of the simulated experiments are shown in the diagrams on this and subsequent pages. First is Scenario 1, with y₂(t) directly observable. For the purpose of elucidation, two versions are shown: first with a “lax” control algorithm that permits blood glucose to drop to below 50 mg/dl during the initial gradient descent (which would be clinically unacceptable), and then a second graph showing much tighter algorithms for safer control, always above 60 mg/dl. The first plots show the convergent dual-control process more clearly, whereas the second shows a more clinically-appropriate version. /medskip

The other scenario presented is Scenario 2, with y₂(t) not directly observable here but reconstructed from y₁(t) and numerical reconstruction of \dot{y}(t). Again, successful control and convergence of medically-important variables are achieved. /medskip

It can be appreciated that further modifications of these basic scenarios to add various more usual clinical characteristics—introduction of a further variable for interstitial fluid glucose (ISFG) monitored by a continuous glucose monitor (CGM) to replace the continuous blood glucose sensor assumption, constraints on the data reporting of the CGM device, introduction of insulin infusion instead of injection, increasing the complexity of the model, et cetera—will all alter the tracking performance of the proposed algorithm, which would then be re-optimised by re-designing the Lyapunov functions and consequent modification of the gradient descent algorithm.

### CONCLUSION

In this paper a particular form of the PSST, exploiting the theoretical reversibility of the direction of convergence, was translated from industrial robotics into a medical context for enhanced clinical care, and a simple implementation was demonstrated for a “minimalist” model of T1D for real-time clinical control under various conditions of partial information. Convergence of the trajectories for observed and estimated glucose was demonstrated during simultaneous control and estimation. The underlying mathematical theory was also presented.

Depending on the complexity of the system equations, available sensors and the design of the Lyapunov function Vₘμ, asymptotic convergence of the trajectories of model and observed variables may then result in either unique estimates of the patient parameters, or else multiple candidates for estimates. If the latter, then this technique may be followed by an additional disambiguation process (i.e. applying a medication control program—additional boluses of insulin—that would have different observable outcomes for the different candidates, thus elucidating which estimates are the most accurate) if desired.

This medical version of the PSST is designed to scale to much more complex models than is represented by the Palumbo-De Gaetano model. It should also be apparent that this approach could be employed clinically for other medical conditions for which adequate PK/PD models are known and appropriate clinical sensors are available to generate time-series data during a medical intervention. The dosing regime of the drug would then be decided, not according to conventional medical dosages, but in real time by the algorithm.

### Variations

The techniques can be applied to any subject, and this includes, but is not limited to patients of human or other mammalian, or non-mammalian species and includes any individual it is desired to examine or treat using the methods of the invention. Suitable subjects include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

It will also be appreciated that the techniques can be used in vitro to examine the reaction of specific samples. Thus for example, the techniques can be used to monitor the reaction of cells to respective environmental conditions, such as combinations of nutrients or the like, and then modify the combination of nutrients, to thereby alter the cells response.

Furthermore, it will be understood that the terms “patient” and “condition”, where used, do not imply that symptoms are present, or that the techniques should be restricted to medical or biological conditions per se. Instead the techniques can be applied to any condition of the subject. Thus, for example, the techniques can be applied to performance subjects, such as athletes, to determine the subject's response to training. This allows a training program to be developed that will be able to prepare the subject for performance events, whilst avoiding overtraining and the like.

Thus, it will be appreciated that the condition of the subject may be the current physical condition, and particularly the readiness for race fitness, with the treatment program being a revised training program specifically directed to the athlete's needs.

In the case of humans, the conditions to which the techniques are most ideally suited include conditions such as:
a) Degenerative diseases such as Parkinson's or Alzheimer's;
b) Disorders involving dopaminergic neurons;
c) Schizophrenia;
d) Bipolar disorders/manic depression;
e) Cardiac disorders;
f) Myasthenia gravis;
g) Neuro-muscular disorders;
h) Cancerous and tumorous cells and related disorders;
i) HIV/AIDS and other immune or auto-immune system disorders;
j) Hepatic disorders;
k) Athletic conditioning;
l) Pathogen related conditions;
m) Viral, bacterial or other infectious diseases;
n) Leukemia;
o) Poisoning, including snakebite and other venom-based disorders;
p) Insulin-dependent diabetes;
q) Clinical trialing of drugs;
r) Any other instances of medication or drug administration to a subject, such that repeated doses are administered over time to maintain drug or ligand concentration to a desired level or within an interval of levels, in the presence of dissipative pharmacokinetic processes such as those of uptake or absorption, distribution or transport, metabolism or elimination;
s) Reconstruction of cardiac rhythms, function, arrhythmia or other cardiac output;
t) Drug-based regulation of arterial pressure;
u) Other disorders or diseases whose significant processes are capable of being reduced to a mathematical model.

However, it will be appreciated that the process can be implemented with respect to any condition for which it is possible to construct a mathematical model of the condition. This is not therefore restricted to medical conditions, although the techniques are ideally suited for the application to conditions such as diseases or other medical disorders.

Persons skilled in the art will appreciate that numerous variations and modifications will become apparent. All such variations and modifications which become apparent to persons skilled in the art, should be considered to fall within the spirit and scope that the invention broadly appearing before described.

1. A method of treating diabetes within a biological subject, the method comprising, in a processing system having a processor and a memory:
a) using the processing system to determine measured subject attributes of the biological subject, the measured subject attributes being periodically measured over a time period and wherein the measured subject attributes include a glucose attribute at least partially indicative of blood glucose levels;
b) using the processing system to determine a base model including one or more equations including at least one non-linear ordinary differential equation or difference equation and wherein the model includes associated variables and parameters including:
one or more state variables representing rapidly changing subject attributes, and including the glucose attribute;
one or more parameters representing slowly changing or constant subject attributes, and including at least one of:
a glucose disappearance rate constant;
an insulin disappearance rate constant;
a plasma glucose concentration increase for a given dosage of glucose administered to the subject; and,
a plasma insulin concentration increase for a given dosage of insulin administered to the subject; and,
one or more control variables representing attributes of a biological response of the subject that can be externally controlled using control inputs provided to the subject, and wherein at least one control input represents treatment that can be provided to the subject, the one or more control variables including at least one dosage of a substance to be administered to the subject;
c) using the processor of the processing system to calculate at least one model value using the base model, the at least one model value being a value of at least one of a state variable, a control variable and a parameter;
d) using the processor of the processing system to compare the measured subject attributes and at least one corresponding model value to determine a difference between the measured subject attributes and the at least one corresponding model value, the difference representing an accuracy of the base model, and the difference being determined by having the processor:
i) derive a subject trajectory representing changes in the measured subject attributes over the time period;
ii) calculate a model trajectory representing changes in the at least one corresponding model value over the time period; and,
iii) perform the comparison by comparing the subject and model trajectories;
e) using the processor of the processing system to modify the base model in accordance with the determined difference, to improve said accuracy of the base model, the base model being modified by modifying at least one of:
i) at least one equation; and,
ii) at least one model value;
f) using the processor of the processing system to repeat steps c) to e) to iteratively modify the base model to thereby generate a subject model representing the condition, the iterative modifying being performed until at least one of:
i) the difference is below a predetermined threshold;
ii) the difference asymptotically approaches an acceptable limit; and,
iii) the difference is minimised;
g) using the subject model to treat a condition within the subject by:
using the model to derive a treatment regime including at least one dosage of a substance to be administered to the subject; and
administering the at least one dosage of the substance to the subject in accordance with the treatment regime to thereby treat the subject, and wherein the substance is at least one of insulin and glucose.

2. A method according to claim 1, wherein the method includes, in the processor of the processing system, iteratively modifying the base model until the model and subject trajectories converge.

3. A method according to claim 1, wherein the iterative process includes:
using external control inputs by administering treatment to the subject to induce a perturbation or agitation of a status of the subject; and,
determining at least one measured subject attribute whilst the perturbation or agitation is induced so that the model can be modified to simulate the application of the control inputs; and,

4. A method according to claim 1, wherein the method includes:
a) using control inputs by administering treatment to the subject to induce at least one of a perturbation and agitation of the subject into a non-equilibrium condition; and,
b) determining at least one measured subject attribute under the non-equilibrium condition.

5. A method according to claim 1, wherein the method includes, in the processor of the processing system, using the model to derive a treatment regime by:
a) forming a linear error equation representing a difference between a desired state of the subject and an actual state; and,
b) constructing a control algorithm including control variable values that minimise the error equation.

6. A method according to claim 1, wherein the method includes, in the processor of the processing system, at least one of:
a) using Lyapunov stability methods to ensure convergence of subject and model behaviour through use of one or more Lyapunov functions; and,
b) using a derivative of one or more Lyapunov functions to impose convergence of subject and model behaviour.

7. A method according to claim 1, wherein the method includes, in the processor of the processing system, modifying the base model using at least one of:
a) model reference adaptive control-based methods;
b) Lyapunov stability-based methods; and,
c) in the event that the subject exhibits mathematically chaotic behaviour, using data obtained from surface-of-section embedding techniques.

8. A method according to claim 1, wherein the method includes, in the processor of the processing system:
a) determining a Lyapunov function;
b) determining a numerical value of a derivative of a Lyapunov function, and
c) using the Lyapunov function to modify at least one model value.

9. A method according to claim 1, wherein the method includes, in the processor of the processing system, at least one of the following:
a) using the existence of a Lyapunov function as the mathematical basis for employing other algorithms to modify at least one model value; and,
b) in the case of chaotic behaviour being exhibited by the subject, using surface-of-section embedding techniques as the mathematical basis for employing other algorithms to modify at least one model value.

10. A method according to claim 9, wherein the method includes, in the processor of the processing system, at least one of:
a) using pattern-finding or optimisation algorithms to at least one of:
i) select one of a number of predetermined Lyapunov functions; and/or,
ii) optimise a Lyapunov function; and/or
iii) optimise the derivative of a Lyapunov function,
b) searching candidate Lyapunov functions to determine a function resulting in the best improvement to the base model; and,
c) at least one of:
i) searching the derivatives of candidate Lyapunov functions to determine a function resulting in the best improvement to the base model; and
ii) employing candidate derivatives without explicitly invoking the underlying Lyapunov function.

11. A method according to claim 10, wherein the method includes, in the processor of the processing system, using pattern-finding or optimisation algorithms to determine a function or related algorithms resulting in the best improvement to the base model.

12. A method according to claim 1, wherein the method includes, in the processor of the processing system and in the instance of mathematically-chaotic behaviour being exhibited by the subject, at least one of the following:
a) using the data obtained from surface-of-section embedding techniques to determine an improvement in the base model within the domain of chaotic behaviour, by modifying at least one of the following:
i) At least one equation; and,
ii) At least one model value; and,
b) using the data obtained from surface-of-section embedding techniques, to determine an improvement in the base model outside the domain of chaotic behaviour, by modifying at least one of the following:
i) At least one equation; and,
ii) At least one model value.

13. A method according to claim 12, wherein the method includes, in the processor of the processing system:
a) selecting a base model from a number of predetermined base models; and,
b) modifying the base model to thereby simulate a condition within the subject.

14. A method according to claim 1, wherein the base model is formed from at least one of:
a) biological components;
b) pharmacological components;
c) pharmacodynamic components; and,
d) pharmacokinetic components.

15. A method according to claim 1, wherein the measured subject attribute is the subject status and the model value is a model output value indicative of the modelled subject status.

16. A method according to claim 1, wherein the subject is at least one of a patient, an animal or an in vitro tissue culture.

17. A method according to claim 1, wherein the treatment regime is indicative of:
a volume of the at least one dosage of the substance;
a concentration of the at least one dosage of the substance;
a time at which the at least one dosage of the substance is administered; and,
a duration over which the at least one dosage of the substance is administered.

18. A method according to claim 1, wherein at least one of:
the one or more state variables include an insulin attribute indicative of blood insulin levels;
the one or more parameters include at least one of:
a length of time for which blood glucose concentrations influence the pancreatic insulin secretion;
an insulin release rate; and,
a blood glucose concentration due to baseline liver glucose release;
the one or more control variables include at least one dosage of glucose administered to the subject;

19. Apparatus for use in treating diabetes within a biological subject, the apparatus comprising a processing system having a processor for performing functions comprising:
a) determining measured subject attributes of the biological subject, the measured subject attributes being periodically measured over a time period and wherein the measured subject attributes include a glucose attribute at least partially indicative of blood glucose levels;
b) determining a base model including one or more equations including at least one non-linear ordinary differential equation or difference equation and wherein the model includes associated variables and parameters including:
one or more state variables representing rapidly changing subject attributes, and including the glucose attribute;
one or more parameters representing slowly changing or constant subject attributes, and including at least one of:
a glucose disappearance rate constant;
an insulin disappearance rate constant;
a plasma glucose concentration increase for a given dosage of glucose administered to the subject; and,
a plasma insulin concentration increase for a given dosage of insulin administered to the subject; and,
one or more control variables representing attributes of a biological response of the subject that can be externally controlled using control inputs provided to the subject, and wherein at least one control input represents medication that can be provided to the subject, the one or more control variables including at least one dosage of a substance to be administered to the subject;
c) calculating at least one model value using the base model, the at least one model value being a value of at least one of a variable, a control variable and a parameter;
d) comparing the measured subject attributes and at least one corresponding model value to determine a difference between the measured subject attributes and the at least one corresponding model value, the different representing an accuracy of the base model, and the difference being determined by having the processor:
i) derive a subject trajectory representing changes in the measured subject attributes over the time period;
ii) calculate a model trajectory representing changes in the at least one corresponding model value over the time period; and,
iii) perform the comparison by comparing the subject and model trajectories;
e) modifying the base model in accordance with the determined difference, to improve said accuracy of the base model, the base the model being modified by modifying at least one of:
i) at least one equation; and,
ii) at least one model value; and,
f) repeating steps c) to e) to iteratively modify the model to thereby generate a subject model representing the condition, the iterative modifying being performed until at least one of:
i) the difference is below a predetermined threshold;
ii) the difference asymptotically approaches an acceptable limit; and,
iii) the difference is minimised;
g) using the subject model to treat the condition within the subject by:
using the model to derive a treatment regime including at least one dosage of a substance to be administered to the subject; and
administering the at least one dosage of the substance to the subject in accordance with the treatment regime to thereby treat the subject, and wherein the substance is at least one of insulin and glucose.

20. A method of treating a condition within a biological subject, the method comprising, in a processing system having a processor and a memory:
a) using the processing system to determine measured subject attributes of the biological subject, the measured subject attributes being measured over a time period;
b) using the processing system to determine a base model including one or more equations including at least one non-linear ordinary differential equation or difference equation and wherein the model includes associated variables and parameters including:
one or more state variables representing rapidly changing subject attributes;
one or more parameters representing slowly changing or constant subject attributes; and,
one or more control variables representing attributes of a biological response of the subject that can be externally controlled using control inputs provided to the subject, and wherein at least one control input represents medication that can be provided to the subject;
c) using the processor of the processing system to calculate at least one model value using the base model, the at least one model value being a value of at least one of a state variable, a control variable and a parameter;
d) using the processor of the processing system to compare the measured subject attributes and at least one corresponding model value to determine a difference between the measured subject attributes and the at least one corresponding model value, the difference representing an accuracy of the base model, and the difference being determined by having the processor:
i) derive a subject trajectory representing changes in the measured subject attributes over the time period;
ii) calculate a model trajectory representing changes in the at least one corresponding model value over the time period; and,
iii) perform the comparison by comparing the subject and model trajectories;
e) using the processor of the processing system to modify the base model in accordance with the determined difference, to improve said accuracy of the base model, the base model being modified by modifying at least one of:
i) at least one equation; and,
ii) at least one model value;
f) using the processor of the processing system to repeat steps c) to e) to iteratively modify the base model to thereby generate a subject model representing the condition, the iterative modifying being performed until at least one of:
i) the difference is below a predetermined threshold;
ii) the difference asymptotically approaches an acceptable limit; and,
iii) the difference is minimised;
and wherein the iterative process includes:
using external control inputs by administering medication to the subject to induce a perturbation or agitation of a status of the subject; and,
determining at least one measured subject attribute whilst the perturbation or agitation is induced so that the model can be modified to simulate the application of the control inputs; and,
g) using the subject model to treat a condition within the subject by:
using the model to derive a medication regime; and
administering medication to the subject in accordance with the medication regime to thereby treat the subject.
